(19) United States
(12) Patent Application Publication
(10) Pub. No.: US 2010/0183592 A1
(43) Pub. Date: Jul. 22, 2010

(54) HUMAN EPO RECEPTOR AGONISTS, COMPOSITIONS, METHODS AND USES FOR PREVENTING OR TREATING GLUCOSE INTOLERANCE RELATED CONDITIONS

(76) Inventors: Ian E. James, Ardmore, PA (US); Kristen Picha, Malvern, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/442,723

(22) PCT Filed: Sep. 28, 2007
(86) PCT No.: PCT/US07/79964

§ 371 Date: Mar. 25, 2009

Related U.S. Application Data
(60) Provisional application No. 60/827,541, filed on Sep. 29, 2006.

(51) Int. Cl. A61K 39/395; C12N 5/071; A61K 38/02; A61P 3/10

(52) U.S. Cl. 424/133.1; 435/325; 514/2; 424/130.1

(57) ABSTRACT

The present invention relates to at least one human EPO receptor agonist methods for preventing or treating glucose intolerance and/or renal disease associated anemia, including therapeutic compositions, methods and devices.

### BACKGROUND OF THE INVENTION

1. Field of the Invention

The present invention relates to at least one human EPO receptor agonist method for preventing or treating glucose intolerance and/or renal disease associated anemia, including therapeutic compositions, methods and devices.

2. Related Art

Certain disease states involve abnormal erythropoiesis. Recombinant human EPO (rHuEPO) is being used therapeutically in a number of countries. In the United States, the U.S. Food and Drug Administration (FDA) has approved rHuEPO's use in treating anemia associated with end-stage renal disease. Patients undergoing hemodialysis to treat this disorder typically suffer severe anemia, caused by the rupture and premature death of erythrocytes as a result of the dialysis treatment. EPO is also useful in the treatment of other types of anemia. For instance, chemotherapy-induced anemia, anemia associated with myelodysplasia, those associated with various congenital disorders, AIDS-related anemia, and prematurity-associated anemia, may be treated with EPO. Additionally, EPO may play a role in other areas, such as helping to more quickly restore a normal hematocrit in bone marrow transplantation patients, in patients preparing for autologous blood transfusions, and in patients suffering from iron overload disorders.

Erythropoietin (EPO) is a glycoprotein hormone composed of 165 amino acids and four carbohydrate chains that functions as the primary regulator of erythropoiesis by binding to a specific receptor on the surface of erythrocyte precursor cells. This binding signals their proliferation and differentiation into mature red blood cells. The erythropoietin receptor is a 484-amino acid glycoprotein with high affinity for erythropoietin. For the erythropoietin receptor, ligand-induced homodimerization may be one of the key event that governs activation.

Erythropoietin has a relatively short half-life. Intravenously administered erythropoietin is eliminated at a rate consistent with first order kinetics with a circulating half-life ranging from approximately 3 to 4 hours in patients with CRF. Within the therapeutic dose range, detectable levels of plasma erythropoietin are maintained for at least 24 hours. After subcutaneous administration of erythropoietin, peak serum levels are achieved within 5-24 hours and decline slowly thereafter.

The anaemia management market is dominated by erythropoiesis-stimulating agents (ESAs) that target the receptor for the growth factor erythropoietin (EPO), which stimulates the production of red blood cells. Recombinant EPOs epoetin-alfa (Epogen; Amgen and Procrit/Eprex; Johnson & Johnson) and epoetin-beta (NeoRecormon/Eprex; Roche) have been on the market for sevaral years.

Amgen's darbepoetin (Aranesp) is a hyperglycosylated variant of EPO (resulting from two amino acid substitutions) and has a longer half-life compared with epoetin, which enables darbepoetin to maintain target haemoglobin levels with less frequent dosing (once every 3-4 weeks with darbepoetin versus once a week with epoetin). Roche's pegylated epoetin CERA (continuous erythropoiesis receptor activator) is undergoing clinical trials and also has a longer half life than EPO. Hematide comprises a dimeric peptide, unrelated in sequence to either native or marketed EPO, which binds to the EPO receptor and stimulates erythropoiesis.

Small peptidomimetics of erythropoietin were identified by several groups through screening of random phage display peptide libraries for affinity to the erythropoietin receptor. These sequences have no homology with erythropoietin. In functional assays several of these peptides showed activity, but only 1/100,000^{th} that of recombinant erythropoietin. Although several attempts have been made to increase the potency of these peptides by preparing covalent dimers or multimers of peptidomimetics, these compounds can be 1,000-10,000 fold less active than erythropoietin on a molar basis and have very short half lives that has made them not suitable for use as therapeutics.

Several EPO receptor agonists are currently in development in for anemia, CHF and other indications:

| Drug | Company | Indication | Status |
|---|---|---|---|
| Erythropoietins | | | |
| Darbepoetin-alfa (Aranesp) | Amgen | Cancer-related anaemia | Phase III |
| | | Congestive heart failure | Phase II |
| CERA (continuous erythropoiesis | Roche | CKD | BLA |
| receptor activator) | | | filed |
| | | CIA | Phase III |
| NE-180 (GlycoPEG-erythropoietin) | Neose | CKD, CIA | Phase I |
| EPO-Fc | Syntonix | CKD, CIA | Phase I |
| AMG 114 (hyperglycosylated analogue of | Amgen | CIA | Phase I |
| darbepoetin) | | | |
| Synthetic erythropoietin receptor agonist | | | |
| Hematide | Affymax/Takeda* | CKD, CIA, anaemia in patients | Phase II |
| | | with PRCA | |
| Erythropoietin replacements | | | |
| FG-2216 | FibroGen | CKD, CIA | Phase II |
| FG-4592 | FibroGen | Anaemia due to iron processing | Phase I |
| | | deficiency | |

(CIA, chemotherapy-induced anaemia; CKD, chronic kidney disease; PEG, polyethylene glycol; PRCA, pure red cell aplasia.)

However, there is a need to provide new uses for EPO receptor agonists, which overcome one more of these and other problems known in the art and to find new indications and treatments.

### SUMMARY OF THE INVENTION

The present invention provides human EPOR agonists, including small molecule agonists, peptide or protein agonists, agonist antibodies or modified immunoglobulins, cleavage products and other specified portions and variants thereof, and nucleic acid, vectors and host cells encoding thereof, as well as EPOR agonist compositions, formulations, combination therapies and the like, encoding or complementary nucleic acids, vectors, host cells, compositions, formulations, devices, and methods of making and using thereof, for preventing or treating glucose intolerance and related conditions, and/or renal disease associated anemia, as described and/or enabled herein, in combination with what is known in the art.

The present invention also provides compositions, methods and devices for preventing or treating glucose intolerance and/or renal disease associated anemia, using at least one isolated EPO Receptor (EPOR) agonist or specified portion or variant as described herein and/or as known in the art.

EPOR agonists are well known in the art and include, but are not limited to, EPO or EPOR agonist (or agonists that have the affect of activating EPOR, e.g., HIF): peptides, proteins, chemical compounds or small molecules and the like, and nucleic acids, vectors and host cells encoding such peptides or proteins, such as but not limited to EPO, modified EPO, EPO protein and small molecule mimetics, EPO or EPOR agonist antibodies or fragments or fusions thereof.

The present invention also provides at least one composition for preventing or treating glucose intolerance and/or renal disease associated anemia, comprising (a) at least one isolated EPOR agonist; and (b) a suitable carrier or diluent. The carrier or diluent can optionally be pharmaceutically acceptable, according to known methods. The composition can optionally further comprise at least one further compound, protein or composition.

The present invention further provides at least one EPOR agonist, specified portion or variant in a method or composition, when administered in a therapeutically effective amount, for modulation, for treating or reducing the symptoms of at least one of glucose intolerance and/or renal disease associated anemia. (See., e.g., The Merck Manual, 17th ed., Merck Research Laboratories, Merck and Co., Whitehouse Station, N.J. (1999), entirely incorporated herein by reference), as needed in many different conditions, such as but not limited to, prior to, subsequent to, or during a related disease or treatment condition, as known in the art.

The present invention also provides at least one composition, device and/or method of delivery of a therapeutically or prophylactically effective amount of at least one EPOR agonist or specified portion or variant, according to the present invention, for preventing or treating glucose intolerance and/or renal disease associated anemia.

The present invention also provides at least one composition comprising (a) an isolated EPOR agonist encoding nucleic acid and/or EPOR agonist as described herein; and (b) a suitable carrier or diluent, for preventing or treating glucose intolerance and/or renal disease associated anemia. The carrier or diluent can optionally be pharmaceutically acceptable, according to known carriers or diluents. The composition can optionally further comprise at least one further compound, protein or composition.

Also provided is a composition comprising at least one isolated human EPO mimetic hinge core mimetibody and at least one pharmaceutically acceptable carrier or diluent. The composition can optionally further comprise an effective amount of at least one compound or protein selected from at least one of a detectable label or reporter, an anti-infective drug, a glucose intolerance related drug, a renal anemia related drug, a gastrointestinal (GI) tract drug, a hormonal drug, a drug for fluid or electrolyte balance, a hematologic drug, A TNF alpha antagonist, a muscle relaxant, a narcotic, a non-steroid anti-inflammatory drug (NTHE), an analgesic, an anesthetic, a sedative, a local anesthetic, a neuromuscular blocker, an antimicrobial, an antipsoriatic, a corticosteriod, an anabolic steroid, a glucose modulator, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

Also provided is a method for diagnosing, preventing or treating glucose intolerance and/or renal disease associated anemia in a cell, tissue, organ or animal, comprising

(a) contacting or administering a composition comprising an effective amount of at least one EPOR agonist with, or to, the cell, tissue, organ or animal. The method can optionally further comprise using an effective amount of 0.00001-500 mg/kilogram to the cells, tissue, organ or animal. The method can optionally further comprise using the contacting or the administrating by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal.

The present invention further provides any invention described herein.

### DESCRIPTION OF THE FIGURES

FIG. 1A-B. Diabetic mice (db/db) were dosed intravenously with CNTO 530 (0.3 mg/kg) or the negative control MMB (lacking a peptide). A. An IPGTT was done 10 minutes after dosing. B. An IPGTT was done 7 days after dosing.

FIG. 2A-B. Diabetic mice (DIO) were dosed intravenously with CNTO 530 (0.3 mg/kg) or the negative control MMB (lacking a peptide). A. An IPGTT was done 10 minutes after dosing. B. An IPGTT was done 7 days after dosing.

FIG. 3. Diabetic mice (DIO) were dosed intravenously with CNTO 530 (0.3 mg/kg) or the negative control MMB (lacking a peptide). Seven days after dosing, the animals were sacrificed and blood.

FIG. 4A-F. Diabetic mice (DIO) were dosed intravenously with CNTO 530 (0.3 mg/kg) or PBS. IPGTTs were done after 10 minutes (A) 7 days (B), 14 days (C), 21 days (D), 28 days (E), 35 days (F).

FIG. 5A-C. Diabetic mice (DIO) were dosed intravenously with CNTO 530 (0.3 mg/kg) or PBS Animals were sacrificed after various times and blood was collected for hematology measurements. Hemoglobin levels are shown A. 21 days B. 28 days and C. 35 days after dosing.

FIG. 6A-C. Diabetic mice (DIO) were dosed intravenously with CNTO 530 (0.3 mg/kg) or PBS. Animals were sacrificed after various times and blood was collected for insulin measurements. Circulating insulin levels are shown A. 21 days B. 28 days and C. 35 days after dosing.

FIG. 7A-B. Diabetic mice (DIO) were dosed intravenously with EPO (0.03, 0.1, 0.3 mg/kg) or PBS. IPGTTs were done 5 days later. (A) Glucose levels throughout the IPGTT (B) Area under the curve from A.

FIG. 8A-B. Diabetic mice (DIO) were dosed intravenously with EPO (0.03, 0.1, 0.3 mg/kg) or PBS. (A) Hemoglobin and (B) Reticulocytes were measured five days later.

FIG. 9A-B. Diabetic mice (DIO) were dosed intravenously with Darbepoetin (0.01, 0.03, 0.1 mg/kg) or PBS. IPGTTs were done 7 days later. (A) Glucose levels throughout the IPGTT (B) Area under the curve from A.

FIG. 10. Diabetic mice (DIO) were dosed intravenously with Darbepoetin (0.01, 0.03, 0.1 mg/kg) or PBS. Fasting blood glucose was measured 7 days later.

FIG. 11A-B. Diabetic mice (DIO) were dosed intravenously with Darbepoetin (0.03, 0.1, 0.3 mg/kg) or PBS. (A) Hemoglobin and (B) Reticulocytes were measured seven days later.

FIG. 12. Fasting blood glucose of wildtype (C57B16 mice) or transgenic mice (heterozygous or homozygous) overexpressing human EPO.

FIG. 13. GTT on wildtype (C57B16 mice) or transgenic mice (heterozygous or homozygous) overexpressing human EPO.

FIG. 14A-B. Diabetic mice (DIO) were dosed intravenously with CNTO 530 (0.3 mg/kg) or PBS. Glucose (A) and insulin levels (B) were measured after an overnight fast and 20 minutes after a glucose challenge.

FIG. 15. The fasting glucose and insulin levels shown in FIG. 14 were used to calculate HOMA.

FIG. 16. Effect of EPO administration on Hemoglobin in patients with baseline HbA1C levels above 6.0

FIG. 17. Effect of EPO administration on Hemoglobin A1C in patients with baseline HbA1C levels above 6.0

FIG. 18. Effect of EPO administration on Fasting blood glucose in patients with baseline HbA1C levels above 6.0.

FIG. 19: Effect of EPO administration on HbA1C levels based on differences in baseline HbA1C level above 6

FIG. 20: Effects of EPO administration and baseline HbA1C levels on 1-2 and 4-6 month HbA1C levels (Longitudinal data)

FIG. 21: Effect of EPO on 1-2 and 4-6 month fasting blood glucose levels in patients for whom data was available at base line (2 months before EPO administration) and in subsequent time sections (1-3 months and 4-6 months).

### DESCRIPTION OF THE INVENTION

The present invention relates to at least one human EPO receptor agonist method for preventing or treating glucose intolerance and/or renal disease associated anemia, including therapeutic compositions, methods and devices.

EPOR agonists are well known in the art and include, but are not limited to, EPO or EPOR agonist (or agonists that have the affect of activating EPOR, e.g., HIF): peptides, proteins, chemical compounds or small molecules and the like, such as but not limited to EPO, modified EPO, EPO protein and small molecule mimetics, EPO or EPOR agonist antibodies or fragments or fusions thereof.

Non limiting examples of how to make and use EPOR agonists are provided by the listed PCT publications relating thereto in the following table, which publications are entirely incorporated herein by reference as they show the state of the art for how to make and use EPO receptor agonists for methods and treatments of the present invention. Non limiting examples of EPO receptor agonists know in the art include EPO, active fragments and mimetics thereof and chemical EPO receptor agonist, which are also disclosed in the following publications which are entirely incorporated herein by reference:

| PCT Publication | Title |
|---|---|
| WO9818926A1 | Circularly Permuted Erythropoietin Receptor Agonists |
| WO06017772A1 | Stable Suspension Formulations Of Erythropoietin Receptor Agonists |
| WO06017773A1 | Stable Particle Formulations Of Erythropoietin Receptor Agonists |
| WO05025606A1 | Long Acting Erythropoietins That Maintain Tissue Protective Activity Of |
| | Endogenous Erythropoietin |
| WO04022577A2 | Long Acting Erythropoietins That Maintain Tissue Protective Activity Of |
| | Endogenous Erythropoietin |
| WO02062843A2 | Modified Erythropoietin (Epo) With Reduced Immunogenicity |
| WO9533057A1 | Hybrid Molecule Of Formula Gm-Csf-L-Epo Or Epo-L-Gm-Csf For |
| | Hematopoietic Stimulation |
| WO04033651A2 | Erythropoietin: Remodeling And Glycoconjugation Of Erythropoietin |
| WO03053997A2 | Methods Of Increasing Endogenous Erythropoietin (Epo) |
| WO0243572A2 | Erythropoietin And Erythropoietin Receptor Expression In Human Cancer |
| WO9928346A1 | Erythropoietin With High Specific Activity |
| WO06055412A1 | Methods Of Treating Erythropoietin-Resistance |
| WO06014466A2 | Novel Carbamylated Epo And Method For Its Production |
| WO06002646A2 | Novel Carbamylated Epo And Method For Its Production |
| WO05121173A1 | Method For Purifying Erythropoietin |
| WO05070451A1 | Pharmaceutical Composition Comprising Non-Glycosylated Erythropoietin |
| WO05063808A1 | Fc-ERYTHROPOIETIN FUSION PROTEIN WITH IMPROVED |
| | PHARMACOKINETICS |
| WO05051327A2 | Glycopegylated Erythropoietin |
| WO05021579A2 | Epo Mimetic Peptides And Fusion Proteins |
| WO04024761A1 | Hasylated Polypeptides, Especially Hasylated Erythropoietin |
| WO03029291A2 | Pegylated And Diglycosylated Erythropoietin |
| WO02085940A2 | New Polynucleotides And Polypeptides Of The Erythropoietin Gene |
| WO0138342A2 | Novel Peptide Dimers As Agonists Of The Erythropoietin (Epo) Receptor, And |
| | Associated Methods Of Synthesis And Use |
| WO0061164A1 | Modulation Of Excitable Tissue Function By Peripherally Administered |
| | Erythropoietin |
| WO9938890A1 | Erythropoietin With Altered Biological Activity |
| WO9911781A1 | Recombinant Human Erythropoietin With Advantageous Glycosylation Profile |
| WO9907401A2 | The Use Of Erythropoietin And Iron Preparations For Producing |
| | Pharmaceutical Combination Preparations For Treating Rheumatic Diseases |
| WO9905268A1 | Production Of Erythropoietin By Endogenous Gene Activation |
| WO9858660A1 | Pharmaceutical Combination Preparations Containing Erythropoietin And |
| | Modified Haemoglobins |
| WO9709996A1 | Pharmaceutical Combination Preparations Containing Erythropoietin And |
| | Ironpreparations |
| WO9635718A1 | Process For Producing Erythropoietin Containing No Animal Proteins |
| WO06062685A2 | Novel Peptides That Bind To The Erythropoietin Receptor |
| WO06060148A2 | Novel Peptides That Bind To The Erythropoietin Receptor |
| WO06029094A2 | Erythropoietin Derivatives With Altered Immunogenicity |
| WO06014349A2 | Method Of Producing Fully Carbamylated Erythropoietin |
| WO05112998A2 | Compositions And Methods For Preventing Erythropoietin-Associated |
| | Hypertension |
| WO05103076A2 | Erythropoietin Protein Variants |
| WO05100403A2 | Antibodies To Erythropoietin Receptor And Uses Thereof |
| WO05099773A1 | Use Of Erythropoietin For Treatment Of Cancer |
| WO05097167A1 | Combination Dosing Regimen For Erythropoietin |
| WO05092369A2 | Conjugates Of Hydroxyethyl Starch And Erythropoietin |
| WO05087804A1 | Erythropoietin Liquid Formulation |
| WO05079232A2 | A METHOD FOR THE PRODUCTION OF AN ERYTHROPOIETIN |
| | ANALOG-HUMAN IgG FUSION PROTEINS IN TRANSGENIC MAMMAL |
| | MILK |
| WO05063809A1 | Nature-Identical Erythropoietin |
| WO05058347A1 | Use Of Erythropoietin In The Treatment Of Disturbances Of Iron Distribution |
| | In Chronic Inflammatory Intestinal Diseases |
| WO05053745A1 | Erythropoietin Solution Formulation |
| WO05032460A2 | Human Epo Mimetic Hinge Core Mimetibodies, Compositions, Methods And |
| | Uses |
| WO05014025A1 | Formulation Of Albumin-Free Erythropoietin |
| WO05001136A1 | Methods And Compositions For Modulating Erythropoietin Expression |
| WO05000340A1 | Synergistic Compositions Comprising Erythropoietin And Succinic Acid (Salt) |
| WO04108681A1 | Nitrogen-Containing Heteroaryl Compounds And Their Use In Increasing |
| | Endogenous Erythropoietin |
| WO04108667A2 | Formation Of Novel Erythropoietin Conjugates Using Transglutaminase |
| WO04108152A1 | Stable, Aqueous Solution Of Human Erythropoietin, Not Containing Serum |
| | Albumin |
| WO04106373A1 | Erythropoietin Conjugate Compounds With Extended Half-Lives |
| WO04101611A2 | Novel Peptides That Bind To The Erythropoietin Receptor |
| WO04101606A2 | Novel Peptides That Bind To The Erythropoietin Receptor |
| WO04091495A2 | Compositions And Methods Related To Production Of Erythropoietin |
| WO04043382A2 | Enhanced Variants Of Erythropoietin And Methods Of Use |
| WO04035603A2 | Erythropoietin Receptor Binding Antibodies |
| WO04006958A1 | Stable Pharmaceutical Composition Comprising Erythropoietin |
| WO04006948A1 | Stable Pharmaceutical Composition Comprising Erythropoietin |
| WO04005323A2 | Affinity Small Molecules For The Epo Receptor |
| WO04002493A1 | Erythropoietin Production Potentiator |
| WO03094858A2 | Ctp-Extended Erythropoietin |
| WO03064664A1 | Physiologically Regulated Erythropoietin-Exprressing Vector For The |
| | Treatment Of Anaemia |
| WO03055526A2 | Erythropoietin Conjugates |
| WO03048210A1 | Fusion Protein Having Enhanced In Vivo Erythropoietin Activity |
| WO03046013A1 | Fusion Protein Having Enhanced In Vivo Activity Of Erythropoietin |
| WO03037273A2 | Method Of Use Of Erythropoietin To Treat Ischemic Acute Renal Failure |
| WO03012432A1 | Erythropoietin And Anti-Tumor Necrosis Factor Alpha Combination Therapy |
| WO02089799A2 | Use Of Dihydropyrazoles To Increase Erythropoietin And Vascularization |
| WO02064085A2 | Treatment Of Neurological Dysfunction Comprising Fructopyranose |
| | Sulfamates And Erythropoietin |
| WO02053580A2 | Protection, Restoration, And Enhancement Of Erythropoietin-Responsive Cells, |
| | Tissues And Organs |
| WO0249673A2 | Erythropoietin Conjugates |
| WO0248194A1 | Fusion Protein Having The Enhanced In Vivo Activity Of Erythropoietin |
| WO0214356A2 | Therapeutic Methods For Treating Subjects With A Recombinant |
| | Erythropoietin Having High Activity And Reduced Side Effects |
| WO0187329A1 | Liquid Pharmaceutical Composition Containing An Erythropoietin Derivate |
| WO0159074A1 | The Production Method Of Transgenic Porcine Producing Human |
| | Erythropoietin And The Transgenic Porcine |
| WO0136489A2 | Erythropoietin Forms With Improved Properties |
| WO0129178A2 | Epo Primary Response Gene 1, Eprg1 |
| WO0107075A2 | Multi-Dose Erythropoietin Formulations |
| WO0102017A2 | Erythropoietin Derivatives |
| WO0067776A1 | Pharmacokinetic And Pharmacodynamic Modeling Of Erythropoietin |
| | Administration |
| WO0061637A1 | Erythropoietin Receptor Antibodies |
| WO0061169A1 | Pharmaceutical Compositions Of Erythropoietin |
| WO0028066A1 | Host Cells Expressing Recombinant Human Erythropoietin |
| WO0027997A1 | Method For The Massive Culture Of Cells Producing Recombinant Human |
| | Erythropoietin |
| WO0027869A1 | Methods Of Purifying Recombinant Human Erythropoietin From Cell Culture |
| | Supernatants |
| WO0027419A1 | Method For Obtaining Lyophilized Pharmaceutical Compositions Of |
| | Recombinant Human Erythropoietin Stable At Room Temperature |
| WO0014261A1 | Production Of Human Erythropoietin |
| WO0009713A1 | Adenoviral Vectors Encoding Erythropoietin And Their Use In Gene Therapy |
| WO9966054A2 | Erythropoietin Analog-Human Serum Albumin Fusion |
| WO9954279A1 | Substituted Amino Acids As Erythropoietin Mimetics |
| WO9952543A2 | Pharmaceutical Compositions Comprising Erythropoietin For Treatment Of |
| | Cancer |
| WO9947151A1 | Peptide Ligands For The Erythropoietin Receptor |
| WO9921966A1 | Erythropoietin-Mediated Neurogenesis |
| WO9906063A1 | Epo Primary Response Gene, Eprg3pt |
| WO9902709A1 | Recombinant Erythropoietin/Immunoglobulin Fusion Proteins |
| WO9823643A1 | Multimeric Erythropoietin With Increased Biological Activity |
| WO9819695A1 | Device And Method For Delivery Of Erythropoietin |
| WO9807442A1 | Sustainedrelease Preparation Comprising Erythropoietin And Polylactide |
| | Coglycolide |
| WO9749729A1 | Polypeptides Mimicking The Activity Of Human Erythropoietin |
| WO9748729A1 | Bivalent Molecules That Form An Activating Complex With An Erythropoietin |
| | Receptor |
| WO9741526A1 | Small Molecule Mimetics Of Erythropoietin |
| WO9727219A1 | Methods For Purification And Use Of Erythropoietin Binding Protein |
| WO9708200A1 | Erythropoietin Binding Protein Useful For Regulation Of Erythropoiesis Andan |
| | Assay For Determination Thereof |
| WO9640749A1 | Compounds And Peptides That Bind To The Erythropoietin Receptor |
| WO9640073A2 | Composition For Sustained Release Of Non-Aggregated Erythropoietin |
| WO9632413A1 | Process For The Purification Of Glycoproteins Like Erythropoietin |
| WO9619573A1 | Recombinant DNA Molecules And Expression Vectors For Erythropoietin |
| WO9618647A1 | Spray Dried Erythropoietin |
| WO9603438A1 | Antibodies Which Activate An Erythropoietin Receptor |
| WO9505469A1 | Human Erythropoietin Receptor Fragment And Antibodies Thereto |
| WO9505465A1 | Erythropoietin Analogs |
| WO9425055A1 | Erythropoietin Analog Compositions And Methods |
| WO9424160A2 | Erythropoietin Muteins With Enhanced Activity |
| WO9417784A1 | Pulmonary Administration Of Erythropoietin |
| WO9402611A2 | Recombinant Human Erythropoietin With Altered Biological Activity |
| WO9325221A1 | Erythropoietin Drug Delivery System |
| WO9305807A2 | Erythropoietin Potentiating Agents And Methods For Their Use |
| WO9118973A1 | Erythropoietin-Dependent Erythroblastoid Mouse Cell Lines |
| WO9111200A1 | Improved Cyclodextrin Based Erythropoietin Formulation |
| WO9106630A1 | Factor-Dependent Hematoploietic Cell Line Exhibiting Epo-Induced |
| | Erythrocyte Maturation |
| WO9105867A1 | Erythropoietin Isoforms |
| WO9008822A1 | Erythropoietin Receptor |
| WO8800241A1 | Human Erythropoietin Gene: High Level Expression In Stably Transfected |
| | Mammalian Cell |
| WO8604068A1 | Homogeneous Erythropoietin |
| WO8603520A1 | Method For The Production Of Erythropoieti |
| WO8503079A1 | HUMAN ERYTHROPOIETIN CDA CLONE |
| WO8403152A1 | Atcc Hb8209 And Its Monoclonal Antibody To Erythropoietin |

Several EPO receptor agonists are currently in development in for anemia, CHF and other indications and can be used as EPO receptor agonists in methods of the present invention:

| Drug | Company | Indication | Status |
|---|---|---|---|
| Erythropoietins | | | |
| Darbepoetin-alfa (Aranesp) | Amgen | Cancer-related anaemia | Phase III |
| | | Congestive heart failure | Phase II |
| CERA (continuous erythropoiesis | Roche | CKD | BLA |
| receptor activator) | | | filed |
| | | CIA | Phase III |
| NE-180 (GlycoPEG-erythropoietin) | Neose | CKD, CIA | Phase I |
| EPO-Fc | Syntonix | CKD, CIA | Phase I |
| AMG 114 (hyperglycosylated analogue of | Amgen | CIA | Phase I |
| darbepoetin) | | | |
| Synthetic erythropoietin receptor agonist | | | |
| Hematide | Affymax/Takeda* | CKD, CIA, anaemia in patients | Phase II |
| | | with PRCA | |
| Erythropoietin replacements | | | |
| FG-2216 | FibroGen | CKD, CIA | Phase II |
| FG-4592 | FibroGen | Anaemia due to iron processing | Phase I |
| | | deficiency | |

Other EPO receptor antagonists of the present invention include antibody fusion proteins that contain EPO receptor agonist peptides, any of which can be used according to the present invention. Non-limiting examples include EPO mimetibodies as disclosed in U.S. patent application Ser. Nos. 10/609,783, filed Jun. 30, 2003 and 10/935,005, filed Sep. 3, 2004, each entirely incorporated herein by reference. Another a non-limiting example of an EPOR agonist that can be used in methods of the present invention, the present invention also provides at least one isolated EPO mimetic hinge core mimetibody or specified portion or variant as described herein and/or as known in the art. The EPO mimetic hinge core mimetibody can optionally comprise at least one CH3 region directly linked with at least one CH2 region directly linked with at least one hinge region or fragment thereof (H), directly linked with an optional linker sequence (L), directly linked to at least one therapeutic peptide (P), optionally further directly linked with at least a portion of at least one variable (V) antibody sequence.

In a preferred embodiment an EPO mimetic hinge core mimetibody comprises formula (I):

((V(m)-P(n)-L(o)-H(p)-CH2(q)-CH3(r))(s),

where V is at least one portion of an N-terminus of an immunoglobulin variable region, P is at least one bioactive peptide, L is polypeptide that provides structural flexibility by allowing the mimietibody to have alternative orientations and binding properties, H is at least a portion of an immunoglobulin variable hinge region, CH2 is at least a portion of an immunoglobulin CH2 constant region, CH3 is at least a portion of an immunoglobulin CH3 constant region, m, n, o, p, q, r, and s can be independently an integer between 0, 1 or 2 and 10, mimicking different types of immunoglobulin molecules, e.g., but not limited to IgG1, IgG2, IgG3, IgG4, IgA, IgM, IgD, IgE, and the like, or combination thereof. The monomer where m=1 can be linked to other monomers by association or covalent linkage, such as, but not limited to, a Cys-Cys disulfide bond or other immunoglobulin sequence. EPO mimetic hinge core mimetibody of the present invention mimics an antibody structure with its inherent properties and functions, while providing a therapeutic peptide and its inherent or acquired in vitro, in vivo or in situ properties or activities. The various portions of the antibody and therapeutic peptide portions of at least one EPO mimetic hinge core mimetibody of the present invention can vary as described herein in combination with what is known in the art.

As used herein, a “EPO mimetic hinge core mimetibody,” “EPO mimetic hinge core mimetibody portion,” or “EPO mimetic hinge core mimetibody fragment” and/or “EPO mimetic hinge core mimetibody variant” and the like mimics, has or simulates at least one ligand binding or at least one biological activity of at least one protein, such as ligand binding or activity in vitro, in situ and/or preferably in vivo, such as but not limited to at least one of SEQ ID NOS:1-30. For example, a suitable EPO mimetic hinge core mimetibody, specified portion or variant of the present invention can bind at least one protein ligand and includes at least one protein ligand, receptor, soluble receptor, and the like. A suitable EPO mimetic hinge core mimetibody, specified portion, or variant can also modulate, increase, modify, activate, at least one protein receptor signaling or other measurable or detectable activity.

Mimetibodies useful in the methods and compositions of the present invention are characterized by suitable affinity binding to protein ligands or receptors and optionally and preferably having low toxicity. In particular, an EPO mimetic hinge core mimetibody, where the individual components, such as the portion of variable region, constant region (without a CH1 portion) and framework, or any portion thereof (e.g., a portion of the J, D or V regions of the variable heavy or light chain; at least a portion of at least one hinge region, the constant heavy chain or light chain, and the like) individually and/or collectively optionally and preferably possess low immunogenicity, is useful in the present invention. The mimetibodies that can be used in the invention are optionally characterized by their ability to treat patients for extended periods with good to excellent alleviation of symptoms and low toxicity. Low immunogenicity and/or high affinity, as well as other undefined properties, may contribute to the therapeutic results achieved. “Low immunogenicity” is defined herein as raising significant HAMA, HACA or HAHA responses in less than about 75%, or preferably less than about 50, 45, 40, 35, 30, 35, 20, 15, 10, 9, 8, 7, 6, 5, 4, 3, 2, and/or 1% of the patients treated and/or raising low titres in the patient treated (less than about 300, preferably less than about 100 measured with a double antigen enzyme immunoassay) (see, e.g., Elliott et al., Lancet 344:1125-1127 (1994)).

### Utility

The isolated nucleic acids of the present invention can be used for production of at least one EPO mimetic hinge core mimetibody, fragment or specified variant thereof, which can be used (or the DNA encoding) to effect in an cell, tissue, organ or animal (including mammals and humans), to modulate, treat, alleviate, help prevent the incidence of, or reduce the symptoms of, at least one condition, selected from, but not limited to, at least one glucose intolerance and/or renal disease associated anemia, as well as other known or specified protein related conditions.

Such a method can comprise administering an effective amount of a composition or a pharmaceutical composition comprising at least one EPO mimetic hinge core mimetibody or specified portion or variant to a cell, tissue, organ, animal or patient in need of such modulation, treatment, alleviation, prevention, or reduction in symptoms, effects or mechanisms. The effective amount can comprise an amount of about 0.0001 to 500 mg/kg per single or multiple administration, or to achieve a serum concentration of 0.0001-5000 μg/ml serum concentration per single or multiple administration, or any effective range or value therein, as done and determined using known methods, as described herein or known in the relevant arts.

### Citations

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2006); Sambrook, et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2006); Colligan et al., Current Protocols in Protein Science, John Wiley & Sons, NY, N.Y., (1997-2006).

### Mimetibodies or EPO Receptor Agonists of the Present Invention

The EPO mimetic hinge core mimetibody can optionally comprise at least one CH3 region directly linked with at least one CH2 region directly linked with at least one portion of at least one hinge region fragment (H) such as comprising at least one core hinge region, directly linked with an optional linker sequence (L), directly linked to at least one therapeutic peptide (P), optionally further directly linked with at least a portion of at least one variable antibody sequence (V). In a preferred embodiment a pair of a CH3-CH2-H-L-V, the pair linked by association or covalent linkage Thus, an EPO mimetic hinge core mimetibody of the present invention mimics an antibody structure with its inherent properties and functions, while providing a therapeutic peptide and its inherent or acquired in vitro, in vivo or in situ properties or activities. The various portions of the antibody and therapeutic peptide portions of at least one EPO mimetic hinge core mimetibody of the present invention can vary as described herein in combination with what is known in the art.

Mimetibodies of the present invention thus provide at least one suitable property as compared to known proteins, such as, but not limited to, at least one of increased half-life, increased activity, more specific activity, increased avidity, increased or decrease off rate, a selected or more suitable subset of activities, less immunogenicity, increased quality or duration of at least one desired therapeutic effect, less side effects, and the like.

Fragments of mimetibodies according to Formula (I) can be produced by enzymatic cleavage, synthetic or recombinant techniques, as known in the art and/or as described herein. Mimetibodies can also be produced in a variety of truncated forms using antibody genes in which one or more stop codons have been introduced upstream of the natural stop site. The various portions of mimetibodies can be joined together chemically by conventional techniques, or can be prepared as a contiguous protein using genetic engineering techniques. For example, a nucleic acid encoding at least one of the constant regions of a human antibody chain can be expressed to produce a contiguous protein for use in mimetibodies of the present invention. See, e.g., Ladner et al., U.S. Pat. No. 4,946,778 and Bird, R. E. et al., Science, 242: 423-426 (1988), regarding single chain antibodies.

As used herein, the term “human mimetibody” refers to an antibody in which substantially every part of the protein (e.g., EPO mimetic peptide, framework, C_{L}, C_{H} domains (e.g., C_{H}2, C_{H}3), hinge, (V_{L}, V_{H})) is expected to be substantially non-immunogenic in humans with only minor sequence changes or variations. Such changes or variations optionally and preferably retain or reduce the immunogenicity in humans relative to non-modified human antibodies, or mimetibodies of the present invention. Thus, a human antibody and corresponding EPO mimetic hinge core mimetibody of the present invention is distinct from a chimeric or humanized antibody. It is pointed out that a human antibody and EPO mimetic hinge core mimetibody can be produced by a non-human animal or cell that is capable of expressing human immunoglobulins (e.g., heavy chain and/or light chain) genes.

In a preferred embodiment, at least one EPO mimetic hinge core mimetibody or other EPO receptor agonist or specified portion or variant of the present invention is produced by at least one cell line, mixed cell line, immortalized cell or clonal population of immortalized and/or cultured cells. Immortalized protein producing cells can be produced using suitable methods. Preferably, the at least one EPO mimetic hinge core mimetibody or other EPO receptor agonist or specified portion or variant is generated by providing nucleic acid or vectors comprising DNA derived or having a substantially similar sequence to, at least one human immunoglobulin locus that is functionally rearranged, or which can undergo functional rearrangement, and which further comprises a mimetibody structure as described herein, e.g., but not limited to Formula (I), wherein portions of C- and N-terminal variable regions can be used for V, hinge regions for H, CH2 for CH2 and CH3 for CH3, as known in the art.

Nucleic Acid Molecules

Using the information provided herein and what is known in the art, such as the nucleotide sequences encoding for various EPO R peptide or protein agonists, a nucleic acid molecule of the present invention encoding at least one EPO R agonist can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combination thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the present invention can include nucleic acid molecules comprising an open reading frame (ORF), optionally with one or more introns, nucleic acid molecules comprising the coding sequence for an EPO mimetic hinge core mimetibody or other EPO receptor agonist or specified portion or variant; and nucleic acid molecules which comprise a nucleotide sequence substantially different from those described above but which, due to the degeneracy of the genetic code, still encode at least one EPO mimetic hinge core mimetibody or other EPO receptor agonist as described herein and/or as known in the art. Of course, the genetic code is well known in the art. Thus, it would be routine for one skilled in the art to generate such degenerate nucleic acid variants that code for specific EPO mimetic hinge core mimetibody or other EPO receptor agonist or specified portion or variants of the present invention. See, e.g., Ausubel, et al., supra, and such nucleic acid variants are included in the present invention.

As indicated herein, nucleic acid molecules of the present invention which comprise a nucleic acid encoding an EPO mimetic hinge core mimetibody or other EPO receptor agonist or specified portion or variant can include, but are not limited to, those encoding the amino acid sequence of an EPO mimetic hinge core mimetibody or other EPO receptor agonist fragment, by itself; the coding sequence for the entire EPO mimetic hinge core mimetibody or other EPO receptor agonist or a portion thereof; the coding sequence for an EPO mimetic hinge core mimetibody or other EPO receptor agonist, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding an EPO mimetic hinge core mimetibody or other EPO receptor agonist or specified portion or variant can be fused to a marker sequence, such as a sequence encoding a peptide that facilitates purification of the fused EPO mimetic hinge core mimetibody or other EPO receptor agonist or specified portion or variant comprising an EPO mimetic hinge core mimetibody or other EPO receptor agonist fragment or portion.

Polynucleotides which Selectively Hybridize to a Polynucleotide as Described Herein

The present invention provides isolated nucleic acids that hybridize under selective hybridization conditions to a polynucleotide disclosed herein, or others disclosed herein, including specified variants or portions thereof. Thus, the polynucleotides of this embodiment can be used for isolating, detecting, and/or quantifying nucleic acids comprising such polynucleotides.

Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 40-99% sequence identity and can be employed to identify orthologous or paralogous sequences.

Optionally, polynucleotides of this invention will encode at least a portion of an EPO mimetic hinge core mimetibody or other EPO receptor agonist or specified portion or variant encoded by the polynucleotides described herein. The polynucleotides of this invention embrace nucleic acid sequences that can be employed for selective hybridization to a polynucleotide encoding an EPO mimetic hinge core mimetibody or other EPO receptor agonist or specified portion or variant of the present invention. See, e.g., Ausubel, supra; Colligan, supra, each entirely incorporated herein by reference.

### Construction of Nucleic Acids

The isolated nucleic acids of the present invention can be made using (a) recombinant methods, (b) synthetic techniques, (c) purification techniques, or combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention—excluding the coding sequence—is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. See, e.g., Ausubel, supra; or Sambrook, supra.

### Recombinant Methods for Constructing Nucleic Acids

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under suitable stringency conditions, to the polynucleotides of the present invention are used to identify the desired sequence in a cDNA or genomic DNA library. The isolation of RNA, and construction of cDNA and genomic libraries, is well known to those of ordinary skill in the art. (See, e.g., Ausubel, supra; or Sambrook, supra).

### Synthetic Methods for Constructing Nucleic Acids

The isolated nucleic acids of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The present invention further provides recombinant expression cassettes comprising a nucleic acid of the present invention. A nucleic acid sequence of the present invention, for example a cDNA or a genomic sequence encoding an EPO mimetic hinge core mimetibody or other EPO receptor agonist or specified portion or variant of the present invention, can be used to construct a recombinant expression cassette that can be introduced into at least one desired host cell. A recombinant expression cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In some embodiments, isolated nucleic acids that serve as promoter, enhancer, or other elements can be introduced in the appropriate position (upstream, downstream or in intron) of a non-heterologous form of a polynucleotide of the present invention so as to up or down regulate expression of a polynucleotide of the present invention. For example, endogenous promoters can be altered in vivo or in vitro by mutation, deletion and/or substitution, as known in the art. A polynucleotide of the present invention can be expressed in either sense or anti-sense orientation as desired. It will be appreciated that control of gene expression in either sense or anti-sense orientation can have a direct impact on the observable characteristics. Another method of suppression is sense suppression. Introduction of nucleic acid configured in the sense orientation has been shown to be an effective means by which to block the transcription of target genes.

### Vectors And Host Cells

The present invention also relates to vectors that include isolated nucleic acid molecules of the present invention, host cells that are genetically engineered with the recombinant vectors, and the production of at least one EPO mimetic hinge core mimetibody or other EPO receptor agonist or specified portion or variant by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced into a cell using suitable known methods, such as electroporation and the like, other known methods include the use of the vector as a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites optionally for at least one of transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one EPO mimetic hinge core mimetibody or other EPO receptor agonist or specified portion or variant of the present invention can be expressed in a modified form, such as a fusion protein, and can include not only secretion signals, but also additional heterologous functional regions. For instance, a region of additional amino acids, particularly charged amino acids, can be added to the N-terminus of an EPO mimetic hinge core mimetibody or other EPO receptor agonist or specified portion or variant to improve stability and persistence in the host cell, during purification, or during subsequent handling and storage. Also, peptide moieties can be added to an EPO mimetic hinge core mimetibody or other EPO receptor agonist or specified portion or variant of the present invention to facilitate purification. Such regions can be removed prior to final preparation of an EPO mimetic hinge core mimetibody or other EPO receptor agonist or at least one fragment thereof. Such methods are described in many standard laboratory manuals, such as Sambrook, (2003) supra, Chapters 17.29-17.42 and 18.1-18.74; Ausubel (2003), supra, Chapters 16, 17 and 18.

Those of ordinary skill in the art are knowledgeable in the numerous expression systems available for expression of a nucleic acid encoding a protein of the present invention.

Illustrative of cell cultures useful for the production of the mimetibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK, HEK293, BHK21 (e.g., ATCC CRL-10), CHO (e.g., ATCC CRL 1610, DG-44) and BSC-1 (e.g., ATCC CRL-26) cell lines, hepG2 cells, P3X63Ag8.653, SP2/0-Ag14, 293 cells, HeLa cells and the like, which are readily available from, for example, American Type Culture Collection, Manassas, Va. Preferred host cells include cells of lymphoid origin such as myeloma and lymphoma cells. Particularly preferred host cells are P3X63Ag8.653 cells (ATCC Accession Number CRL-1580) and SP2/0-Ag14 cells (ATCC Accession Number CRL-1851).

Expression vectors for these cells can include one or more of the following expression control sequences, such as, but not limited to an origin of replication; a promoter (e.g., late or early SV40 promoters, the CMV promoter (e.g., U.S. Pat. Nos. 5,168,062; 5,385,839), an HSV tk promoter, a pgk (phosphoglycerate kinase) promoter, an EF-1 alpha promoter (e.g, U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Purification of an EPO Mimetic Hinge Core Mimetibody or Other EPO Receptor Agonist or Specified Portion or Variant Thereof

An EPO mimetic hinge core mimetibody or other EPO receptor agonist or specified portion or variant can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2006), e.g., Chapters 1, 4, 6, 8, 9, 10, each entirely incorporated herein by reference.

Mimetibodies or specified portions or variants of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the EPO mimetic hinge core mimetibody or other EPO receptor agonist or specified portion or variant of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

Mimetibodies, Specified Fragments and/or Variants

The isolated mimetibodies of the present invention comprise an EPO mimetic hinge core mimetibody or other EPO receptor agonist or specified portion or variant encoded by any one of the polynucleotides of the present invention as discussed more fully herein, or any isolated or prepared EPO mimetic hinge core mimetibody or other EPO receptor agonist or specified portion or variant thereof.

Preferably, the EPO mimetic hinge core mimetibody or other EPO receptor agonist or ligand-binding portion or variant binds at least one EPO protein ligand or receptor, and, thereby provides at least one EPO biological activity of the corresponding protein or a fragment thereof. Different therapeutically or diagnostically significant proteins are well known in the art and suitable assays or biological activities of such proteins are also well known in the art.

Non-limiting examples of suitable EPO mimetic peptides for this invention appear in Table 1 below. These peptides can be prepared by methods disclosed and/or known in the art. Single letter amino acid abbreviations are used in most cases. The X in these sequences (and throughout this specification, unless specified otherwise in a particular instance) means that any of the 20 naturally occurring or known amino acid residues or know derivatives thereof may be present, or any know modified amino acid thereof. Any of these peptides may be linked in tandem (i.e., sequentially), with or without linkers, and a few tandemlinked examples are provided in the table. Linkers are listed as “Δ” and may be any of the linkers described herein. Tandem repeats and linkers are shown separated by dashes for clarity. Any peptide containing a cysteinyl residue may optionally be cross-linked with another Cys-containing peptide, either or both of which may be linked to a vehicle. A few crosslinked examples are provided in the table. Any peptide having more than one Cys residue may form an intrapeptide disulfide bond, as well; see, for example, EPO-mimetic peptides in Table 1. A few examples of intrapeptide disulfide-bonded peptides are specified in the table. Any of these peptides may be derivatized as described herein, and a few derivatized examples are provided in the table. For derivatives in which the carboxyl terminus may be capped with an amino group, the capping amino group is shown as —NH₂. For derivatives in which amino acid residues are substituted by moieties other than amino acid residues, the substitutions are denoted by a δ, which signifies any of the moieties known in the art, e.g., as described in Bhatnagar et al. (1996), J. Med. Chem. 39: 3814-9 and Cuthbertson et al. (1997), J. Med. Chem. 40:2876-82, which are entirely incorporated by reference. The J substituent and the Z substituents (Z₅, Z₆, . . . Z₄₀) are as defined in U.S. Pat. Nos. 5,608,035, 5,786,331, and 5,880,096, which are entirely incorporated herein by reference. For the EPO-mimetic sequences (Table 1), the substituents X₂ through X₁₁ and the integer “n” are as defined in WO 96/40772, which is entirely incorporated by reference. Residues appearing in boldface are D-amino acids, but can be optionally L-amino acids. All peptides are linked through peptide bonds unless otherwise noted. Abbreviations are listed at the end of this specification. In the “SEQ ID NO.” column, “NR” means that no sequence listing is required for the given sequence.

TABLE 1
EPO-mimetic peptide sequences

| Sequence/structure | SEQ ID NO: |
|---|---|
| YXCXXGPXTWXCXP | 1 |
| YXCXXGPXTWXCXP-YXCXXGPXTWXCXP | 2 |
| YXCXXGPXTWXCXP-Λ-YXCXXGPXTWXCXP | 3 |
| 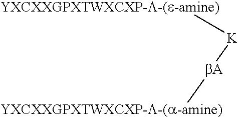 | 4 |
| GGTYSCHFGPLTWVCKPQGG | 5 |
| GGDYHCRMGPLTWVCKPLGG | 6 |
| GGVYACRMGPITWVCSPLGG | 7 |
| VGNYMCHFGPITWVCRPGGG | 8 |
| GGLYLCRFGPVTWDCGYKGG | 9 |
| GGTYSCHFGPLTWVCKPQGG | 10 |
| GGTYSCHFGPLTWVCKPQGG-GGTYSCHFGPLTWVCKPQGG | 11 |
| GGTYSCHFGPLTWVCKPQGG-Λ-GGTYSCHFGPLTWVCKPQGG | 12 |
| GGTYSCHFGPLTWVCKPQGGSSK | 13 |
| GGTYSCHFGPLTWVCKPQGGSSK-GGTYSCHFGPLTWVCKPQGGSSK | 14 |
| GGTYSCHFGPLTWVCKPQGGSSK-Λ-GGTYSCHFGPLTWVCKPQGGSSK | 15 |
| 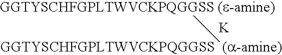 | 16 |
| GGTYSCHFGPLTWVCKPQGGSSK (-Λ-biotin) | 17 |
| CX₄X₅GPX₆TWX₇C | 18 |
| GGTYSCHGPLTWVCKPQGG | 19 |
| VGNYMAHMGPITWVCRPGG | 20 |
| GGPHHVYACRMGPLTWIC | 21 |
| GGTYSCHFGPLTWVCKPQ | 22 |
| GGLYACHMGPMTWVCQPLRG | 23 |
| TIAQYICYMGPETWECRPSPKA | 24 |
| YSCHFGPLTWVCK | 25 |
| YCHFGPLTWVC | 26 |
| X₃X₄X₅GPX₆TWX₇X₈ | 27 |
| YX₂X₃X₄X₅GPX₆TWX₇X₈ | 28 |
| X₁YX₂X₃X₄X₅GPX₆X₇X₈X₉X₁₀X₁₁ | 29 |
| X₁YX₂CX₄X₅GPX₆TWX₇CX₉X₁₀X₁₁ | 30 |
| GGLYLCRFGPVTWDCGYKGG | 31 |
| GGTYSCHFGPLTWVCKPQGG | 32 |
| VGNYMCHFGPITWVCRPGGG | 33 |
| GGVYACRMGPITWVCSPLGG | 34 |
| TIAQYICYMGPETWECRPSPKA | 35 |
| YSCHFGPLTWVCK | 36 |
| YCHFGPLTWVC | 37 |
| SCHFGPLTWVCK | 38 |
| (AX₂)ₙX₃X₄X₅GPX₆TWX₇X₈ | 39 |

EPO biological activities are well known in the art. See, e.g., Anagnostou A et al Erythropoietin has a mitogenic and positive chemotactic effect on endothelial cells. Proceedings of the National Academy of Science (USA) 87: 5978-82 (1990); Fandrey J and Jelkman W E Interleukin 1 and tumor necrosis factor-alpha inhibit erythropoietin production in vitro. Annals of the New York Academy of Science 628: 250-5 (1991); Geissler K et al Recombinant human erythropoietin: A multipotential hemopoietic growth factor in vivo and in vitro. Contrib. Nephrol. 87: 1-10 (1990); Gregory C J Erythropoietin sensitivity as a differentiation marker in the hemopoietic system. Studies of three erythropoietic colony responses in culture. Journal of Cellular Physiology 89: 289-301 (1976); Jelkman W et al Monokines inhibiting erythropoietin production in human hepatoma cultures and in isolated perfused rat kidneys. Life Sci. 50: 301-8 (1992); Kimata H et al Human recombinant erythropoietin directly stimulates B cell immunoglobulin production and proliferation in serum-free medium. Clinical and Experimental Immunology 85: 151-6 (1991); Kimata H et al Erythropoietin enhances immunoglobulin production and proliferation by human plasma cells in a serum-free medium. Clin. Immunology Immunopathol. 59: 495-501 (1991); Kimata H et al Effect of recombinant human erythropoietin on human IgE production in vitro Clinical and Experimental Immunology 83: 483-7 (1991); Koury M J and Bondurant M C Erythropoietin retards DNA breakdown and prevents programmed cell death in erythroid progenitor cells. Science 248: 378-81 (1990); Lim V S et al Effect of recombinant human erythropoietin on renal function in humans. Kidney International 37: 131-6 (1990); Mitjavila M T et al Autocrine stimulation by erythropoietin and autonomous growth of human erythroid leukemic cells in vitro. Journal of Clinical Investigation 88: 789-97 (1991); Andre M et al Performance of an immunoradiometric assay of erythropoietin and results for specimens from anemic and polycythemic patients. Clinical Chemistry 38: 758-63 (1992); Hankins W D et al Erythropoietin-dependent and erythropoietin-producing cell lines. Implications for research and for leukemia therapy. Annals of the New York Academy of Science 554: 21-8 (1989); Kendall R G T et al Storage and preparation of samples for erythropoietin radioimmunoassay. Clin. Lab. Haematology 13: 189-96 (1991); Krumvieh D et al Comparison of relevant biological assays for the determination of biological active erythropoietin. Dev. Biol. Stand. 69: 15-22 (1988); Ma D D et al Assessment of an EIA for measuring human serum erythropoietin as compared with RIA and an in-vitro bioassay. British Journal of Haematology 80: 431-6 (1992); Noe G et al A sensitive sandwich ELISA for measuring erythropoietin in human serum British Journal of Haematology 80: 285-92 (1992); Pauly J U et al Highly specific and highly sensitive enzyme immunoassays for antibodies to human interleukin 3 (IL3) and human erythropoietin (EPO) in serum. Behring Institut Mitteilungen 90: 112-25 (1991); Sakata S and Enoki Y Improved microbioassay for plasma erythropoietin based on CFU-E colony formation. Ann. Hematology 64: 224-30 (1992); Sanengen T et al Immunoreactive erythropoietin and erythropoiesis stimulating factor(s) in plasma from hypertransfused neonatal and adult mice. Studies with a radioimmunoassay and a cell culture assay for erythropoietin. Acta Physiol. Scand. 135: 11-6 (1989); Widness J A et al A sensitive and specific erythropoietin immunoprecipitation assay: application to pharmacokinetic studies. Journal of Lab. Clin. Med. 119: 285-94 (1992); for further information see also individual cell lines used in individual bioassays. Each of the above references are entirely incorporated herein by reference. EPO can be assayed by employing cell lines such as HCD57, NFS-60, TF-1 and UT-7, which respond to the factor. EPO activity can be assessed also in a Colony formation assay by determining the number of CFU-E from bone marrow cells. An alternative and entirely different detection method is RT-PCR quantitation of cytokines.

An EPO mimetic hinge core mimetibody or other EPO receptor agonist, or specified portion or variant thereof, that partially or preferably substantially provides at least one biological activity of at least one protein or fragment, can bind the protein or fragment ligand and thereby provide at least one activity that is otherwise mediated through the binding of protein to at least one protein ligand or receptor or through other protein-dependent or mediated mechanisms. As used herein, the term “EPO mimetic hinge core mimetibody or other EPO receptor agonist activity” refers to an EPO mimetic hinge core mimetibody or other EPO receptor agonist that can modulate or cause at least one protein-dependent activity by about 20-10,000%, preferably by at least about 60, 70, 80, 90, 91, 92, 93, 94, 95, 96, 97, 98, 99, 100, 110, 120, 130, 140, 150, 160, 170, 180, 190, 200, 250, 300, 350, 400, 450, 500, 550, 600, 700, 800, 900, 1000, 2000, 3000, 4000, 5000, 6000, 7000, 8000, 9000% or more depending on the assay.

The capacity of an EPO mimetic hinge core mimetibody or other EPO receptor agonist or specified portion or variant to provide at least one protein-dependent activity is preferably assessed by at least one suitable protein biological assay, as described herein and/or as known in the art. A human EPO mimetic hinge core mimetibody or other EPO receptor agonist or specified portion or variant of the invention can be similar to any class (IgG, IgA, IgM, etc.) or isotype and can comprise at least a portion of a kappa or lambda light chain. In one embodiment, the human EPO mimetic hinge core mimetibody or other EPO receptor agonist or specified portion or variant comprises an IgG heavy chain variable fragment, hinge region, CH2 and CH3, for example, at least one of isotypes, IgG1, IgG2, IgG3 or IgG4.

At least one EPO mimetic hinge core mimetibody or other EPO receptor agonist or specified portion or variant of the invention binds at least one specified ligand specific to at least one protein, subunit, fragment, portion or any combination thereof. The at least one EPO mimetic peptide of at least one EPO mimetic hinge core mimetibody or other EPO receptor agonist, specified portion or variant of the present invention can optionally bind at least one specified ligand epitope of the ligand. The binding epitope can comprise any combination of at least one amino acid sequence of at least 1-3 amino acids to the entire specified portion of contiguous amino acids of the sequences selected from the group consisting of a protein ligand, such as an EPO receptors or portion thereof.

Such mimetibodies can be prepared by joining together the various portions of Formula (I) of the EPO mimetic hinge core mimetibody or other EPO receptor agonist using known techniques, by preparing and expressing at least one (i.e., one or more) nucleic acid molecules that encode the EPO mimetic hinge core mimetibody or other EPO receptor agonist, using known techniques of recombinant DNA technology or by using any other suitable method, such as chemical synthesis.

Mimetibodies that bind to human EPO ligands or receptors and that comprise at least a one portion defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. The EPO mimetic hinge core mimetibody or other EPO receptor agonist, specified portion or variant can be expressed using the encoding nucleic acid or portion thereof in a suitable host cell.

Preferably, such mimetibodies or ligand-binding fragments thereof can bind human EPO ligands or receptors with high affinity (e.g., K_{D} less than or equal to about 10⁻⁷ M) Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g, charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

### Amino Acid Codes

The amino acids that make up mimetibodies or specified portions or variants of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994), as presented in Table

TABLE 2

| SINGLE | THREE | | |
|---|---|---|---|
| LETTER | LETTER | | THREE NUCLEOTIDE |
| CODE | CODE | NAME | CODON(S) |
| A | Ala | Alanine | GCA, GCC, GCG, GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, CUC, |
| | | | CUG, CUU |
| M | Met | Methionine | AUG |
| N | Asn | Asparagine | AAC, AAU |
| P | Pro | Proline | CCA, CCC, CCG, CCU |
| Q | Gln | Glutamine | CAA, CAG |
| R | Arg | Arginine | AGA, AGG, CGA, CGC, |
| | | | CGG, CGU |
| S | Ser | Serine | AGC, AGU, UCA, UCC, |
| | | | UCG, UCU |
| T | Thr | Threonine | ACA, ACC, ACG, ACU |
| V | Val | Valine | GUA, GUC, GUG, GUU |
| W | Trp | Tryptophan | UGG |
| Y | Tyr | Tyrosine | UAC, UAU |

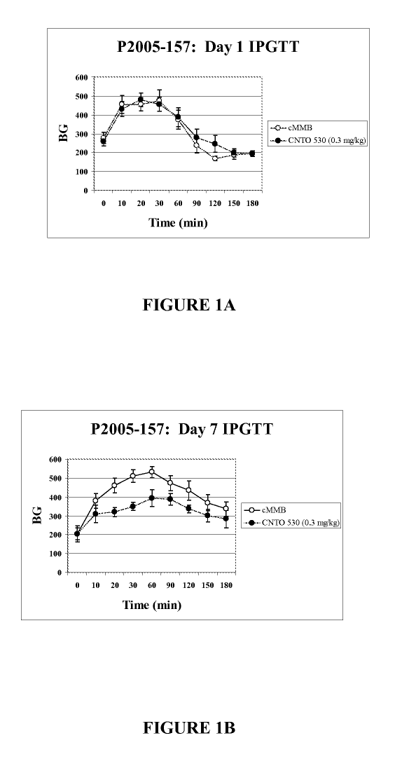

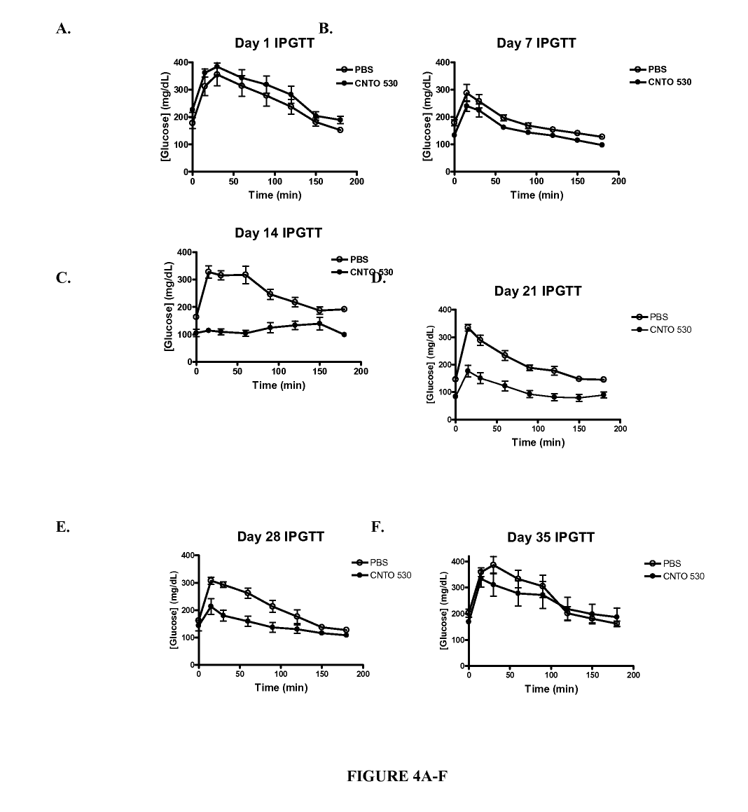

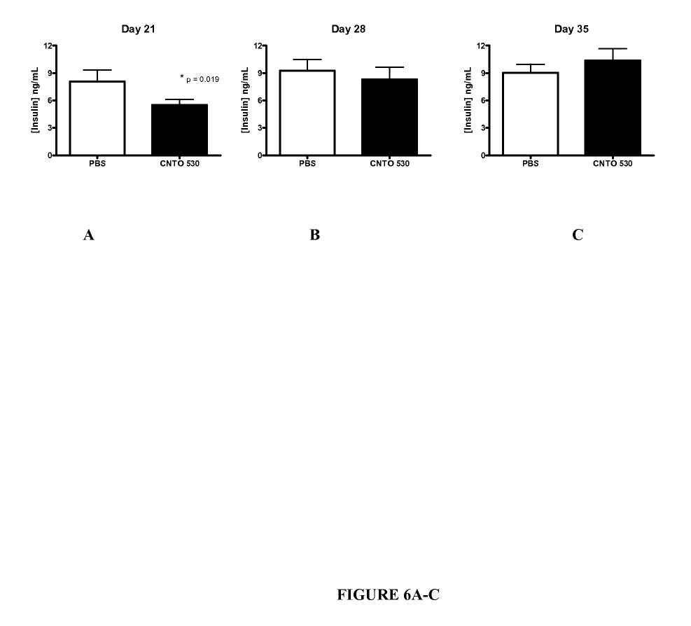

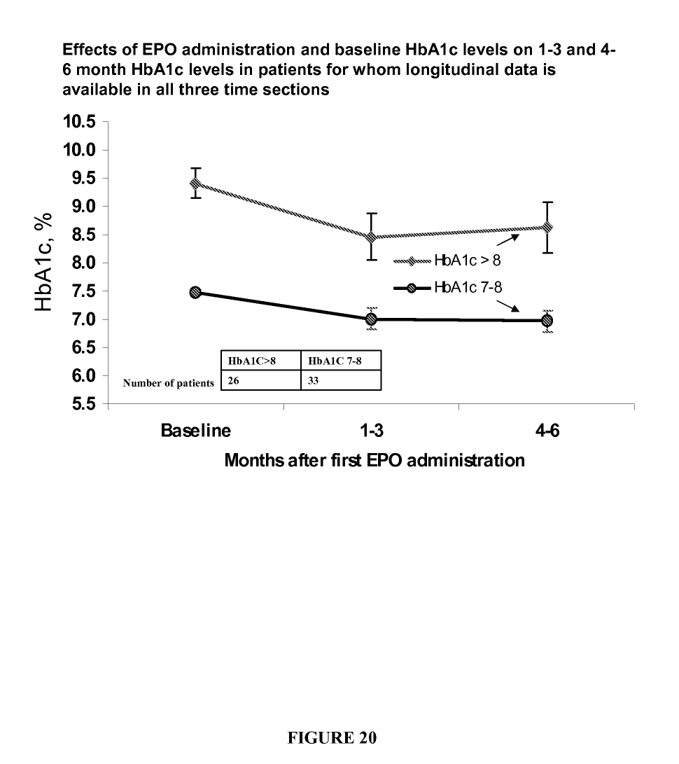

An EPO mimetic hinge core mimetibody or other EPO receptor agonist or specified portion or variant of the present invention can include one or more amino acid substitutions, deletions or additions, either from natural mutations or human manipulation, as specified herein. Such or other sequences that can be used in the present invention, include, but are not limited to the following sequences presented in Table 3, as further described in FIGS. 1-42 of U.S. provisional application 60/507,349, filed 30 Sep. 2003, entirely incorporated by reference herein, corresponding to FIGS. 1-41 of PCT Appl. No. US04/19783, filed Jun. 17, 2004, entirely incorporated herein by reference, with corresponding SEQ ID NOS:31-72. These referenced FIGS. 1-42 (SEQ ID NOS:31-72), or FIGS. 1-41 of PCT US04/19783, show examples of heavy/light chain variable/constant region sequences, frameworks/subdomains and substitutions, portions of which can be used in Ig derived proteins of the present invention, as taught herein.

TABLE 3

| SEQ ID | | | AA | REGIONS | | | | | | |
|---|---|---|---|---|---|---|---|---|---|---|
| NO | | | NO | FR1 | CDR1 | FR2 | CDR2 | FR3 | CDR3 | FR4 |
| 31 | Heavy | Vh1 | 125 | 1-31 | 32 | 33-46 | 47 | 48-79 | 80 | 81-125 |
| 32 | chain | Vh2 | 124 | 1-30 | 31 | 32-45 | 46 | 47-78 | 79 | 80-124 |
| 33 | variable | Vh3a | 100 | 1-31 | 32 | 33-46 | 47 | 48-79 | 80 | 81-100 |
| 34 | region | Vh3b | 102 | 1-30 | 31 | 32-45 | 46 | 47-78 | 79 | 80-102 |
| 35 | | Vh3c | 101 | 1-30 | 31 | 32-45 | 46 | 47-79 | 80 | 81-101 |
| 36 | | Vh4 | 108 | 1-33 | 34 | 35-48 | 49 | 50-81 | 82 | 83-108 |
| 37 | | Vh5 | 132 | 1-31 | 32 | 33-46 | 47 | 48-79 | 80 | 81-132 |
| 38 | | Vh6 | 125 | 1-30 | 31 | 32-45 | 46 | 47-78 | 79 | 80-125 |
| 39 | | Vh7 | 91 | 1-30 | 31 | 32-45 | 46 | 47-78 | 79 | 80-91 |
| 40 | Light chain | κ1-4 | 93 | 1-24 | 25 | 26-40 | 41 | 42-73 | 74 | 75-93 |
| 41 | variable | κ2 | 92 | 1-23 | 24 | 25-39 | 40 | 41-72 | 73 | 74-92 |
| 42 | region | κ3 | 91 | 1-23 | 24 | 25-39 | 40 | 41-72 | 73 | 74-91 |
| 43 | | κ5 | 85 | 1-23 | 24 | 25-39 | 40 | 41-72 | 73 | 74-85 |
| 44 | | κ new1 | 79 | 1-17 | 18 | 19-33 | 34 | 35-66 | 67 | 68-79 |
| 45 | | κ new2 | 77 | 1-15 | 16 | 17-31 | 32 | 33-64 | 65 | 66-77 |
| 46 | | κ new3 | 95 | 1-24 | 25 | 26-40 | 41 | 42-73 | 74 | 75-95 |
| 47 | | λ1a | 98 | 1-22 | 23 | 24-38 | 39 | 40-71 | 72 | 73-98 |
| 48 | | λ1b | 99 | 1-23 | 24 | 25-39 | 40 | 41-72 | 73 | 74-99 |
| 49 | | λ2 | 99 | 1-22 | 23 | 24-38 | 39 | 40-71 | 72 | 73-99 |
| 50 | | λ3a | 107 | 1-22 | 23 | 24-38 | 39 | 40-71 | 72 | 73-107 |
| 51 | | λ3b | 93 | 1-22 | 23 | 24-39 | 40 | 41-72 | 73 | 74-93 |
| 52 | | λ3c | 98 | 1-22 | 23 | 24-38 | 39 | 40-71 | 72 | 73-98 |
| 53 | | λ3e | 98 | 1-22 | 23 | 24-38 | 39 | 40-71 | 72 | 73-98 |
| 54 | | λ4a | 94 | 1-22 | 23 | 24-38 | 39 | 40-71 | 72 | 73-94 |
| 55 | | λ4b | 95 | 1-22 | 23 | 24-38 | 39 | 40-71 | 72 | 73-95 |
| 56 | | λ5 | 88 | 1-22 | 23 | 24-39 | 40 | 41-74 | 75 | 76-88 |
| 57 | | λ6 | 101 | 1-22 | 23 | 24-38 | 39 | 40-73 | 74 | 75-101 |
| 58 | | λ7 | 89 | 1-22 | 23 | 24-38 | 39 | 40-71 | 72 | 73-89 |
| 59 | | λ8 | 89 | 1-22 | 23 | 24-38 | 39 | 40-71 | 72 | 73-89 |
| 60 | | λ9 | 91 | 1-22 | 23 | 24-38 | 39 | 40-79 | 80 | 81-91 |
| 61 | | λ10 | 87 | 1-22 | 23 | 24-38 | 39 | 40-71 | 72 | 73-87 |
| SEQ ID | | | AA | REGIONS | | | | | | |
| NO | | | NO | CH1 | hinge1 | hinge2 | hinge3 | hinge4 | CH2 | CH3 |
| 62 | Heavy | IgA1 | 354 | 1-102 | 103-121 | | | | 122-222 | 223-354 |
| 63 | chain | IgA2 | 340 | 1-102 | 103-108 | | | | 109-209 | 210-340 |
| 64 | constant | IgD | 384 | 1-101 | 102-135 | 136-159 | | | 160-267 | 268-384 |
| 65 | region | IgE | 497 | 1-103 | | | | | 104-210 | 211-318 |
| 66 | | IgG1 | 339 | 1-98 | 99-113 | | | | 114-223 | 224-339 |
| 67 | | IgG2 | 326 | 1-98 | 99-110 | | | | 111-219 | 220-326 |
| 68 | | IgG3 | 377 | 1-98 | 99-115 | 116-130 | 131-145 | 146-160 | 161-270 | 271-377 |
| 69 | | IgG4 | 327 | 1-98 | 99-110 | | | | 111-220 | 221-327 |
| 70 | | IgM | 476 | 1-104 | | | | | 105-217 | 218-323 |
| 71 | Light chain | Igκc | 107 | | | | | | | |
| 72 | constant | Igλc | 107 | | | | | | | |
| | region | | | | | | | | | |

Of course, the number of amino acid substitutions a skilled artisan would make depends on many factors, including those described above. Generally speaking, the number of amino acid substitutions, insertions or deletions for at least one of an EPO mimetic hinge core mimetibody or other EPO receptor agonist will not be more than 40, 30, 20, 19, 18, 17, 16, 15, 14, 13, 12, 11, 10, 9, 8, 7, 6, 5, 4, 3, 2, 1 amino acids, such as 1-30 or any range or value therein, as specified herein.

The following description of the components of an EPO hinge core mimetibody of the present invention is based on the use of the formula I of the present invention,

((V(m)-P(n)-L(o)-H(p)-CH2(q)-CH3(r))(s),

where V is at least one portion of an N-terminus of an immunoglobulin variable region, P is at least one bioactive peptide, L is at least one linker polypeptide H is at least one portion of at least one immunoglobulin hinge region, CH2 is at least a portion of an immunoglobulin CH2 constant region, CH3 is at least a portion of an immunoglobulin CH3 constant region, m, n, o, p, q, r and s are independently an integer between 0, 1 or 2 and 10, mimicking different types of immunoglobulin molecules, e.g., but not limited to IgG1, IgG2, IgG3, IgG4, IgA, IgM, IgD, IgE, and the like, or any subclass thereof, or any combination thereof.

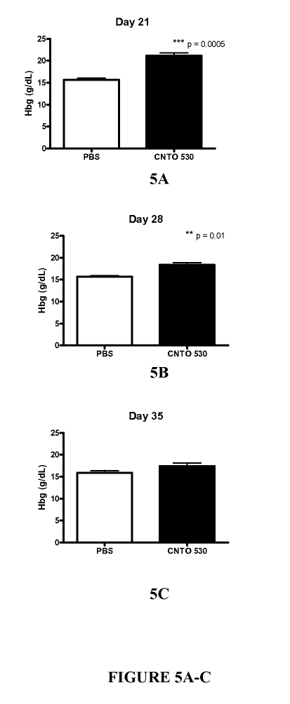

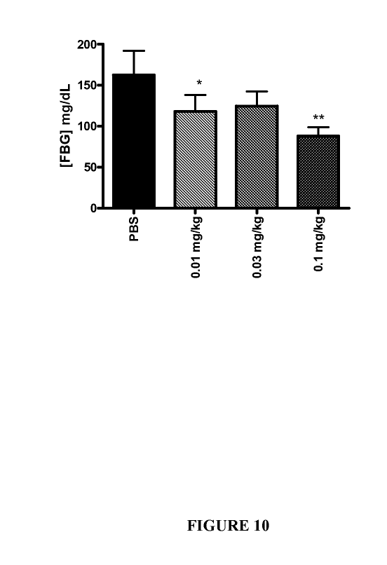

In hinge core mimetibodies of the present invention, the optional N-terminal V portion can comprise 1-20 amino acids of at least one heavy chain variable framework 1 (FR1) region, e.g., as presented in FIGS. 1-9 (SEQ ID NOS:31-39) or at least one LC variable region, e.g., as presented in FIGS. 10-31 (SEQ ID NOS:40-61), of U.S. provisional application 60/507,349, filed 30 Sep. 2003, entirely incorporated by reference herein, corresponding to FIGS. 1-41 of PCT Appl. No. US04/19783, filed Jun. 17, 2004, entirely incorporated herein by reference, including substitutions, deletions or insertions as presented in these Figures, with those of FIGS. 5, 6, and 8 preferred. Also preferred are variable sequences that comprise the sequence Q-X-Q.

The P portion can comprise at least one any therapeutic peptide as known in the art or as described herein, such as, but not limited to those presented in Table 1, SEQ ID NOS:1-30, or as known in the art, or any combination or consensus sequence thereof, or any fusion protein thereof.

The optional linker sequence can be any suitable peptide linker as known in the art. Preferred sequence include any combination of G and S, e.g., X1-X2-X3-X4-Xn, where X can be G or S, and n can be 5-30. Non-limiting examples include, GS, GGGS, GSGGGS, GSGGGSGG, and the like.

In the present invention, the CH1 portion is not used and a variable number of amino acids from the N-terminus of the hinge region are deleted, e.g., as referenced to FIGS. 1-42 of U.S. provisional application 60/507,349, filed 30 Sep. 2003, entirely incorporated by reference herein, corresponding to FIGS. 1-41 of PCT Appl. No. US04/19783, filed Jun. 17, 2004, entirely incorporated herein by reference, and Table 3. The variable number of amino acids used for the hinge core portion of a mimetibody of the present invention include, but are not limited to, deletion of any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 1-3, 2-5, 2-7, 2-8, 3-9, 4-10, 5-9, 5-10, 5-15, 10-20, 2-30, 20-40, 10-50, or any range or value therein, of the N-terminal amino acids of at least one hinge region, e.g., as presented in FIGS. 32-40 of U.S. provisional application 60/507,349, filed 30 Sep. 2003, entirely incorporated by reference herein, corresponding to FIGS. 1-41 of PCT Appl. No. US04/19783, filed Jun. 17, 2004, entirely incorporated herein by reference, or Table 3 above, e.g., but not limited to, deletion of any to all of the amino acids 99-101 to 105-157 of amino acids 99-105, 99-108, 99-111, 99-112, 99-113, 99-114, 99-115, 99-119, 99-125, 99-128, 99-134, 99-140, 99-143, 99-149, 99-155 and 99-158 of FIGS. 32-40 of U.S. provisional application 60/507,349, filed 30 Sep. 2003, entirely incorporated by reference herein, corresponding to FIGS. 1-41 of PCT Appl. No. US04/19783, filed Jun. 17, 2004, entirely incorporated herein by reference, corresponding to SEQ ID NOS:62-70, including the substitutions, insertions or deletions described in FIGS. 32-40 of U.S. provisional application 60/507,349, filed 30 Sep. 2003, entirely incorporated by reference herein, corresponding to FIGS. 1-41 of PCT Appl. No. US04/19783, filed Jun. 17, 2004, entirely incorporated herein by reference. In preferred embodiments, a hinge core regions of the present invention includes a deletion of the N-terminous of the hinge region to provide a hinge core region that includes a deletion up to but not including a Cys residue or up to but not including a sequence Cys-Pro-Xaa-Cys. In further preferred embodiment, such hinge core sequences used in a hinge core mimetibody of the present invention include amino acids 109-113 or 112-113 (SEQ ID NO:66) (IgG1); 105-110 or 109-110 (SEQ ID NO:67) (IgG2); 111-160, 114-160, 120-160, 126-160, 129-160, 135-160, 141-160, 144-160, 150-160, 156-160 and 159-160 (SEQ ID NO:68) (IgG3); or 106-110 or 109-110 (SEQ ID NO:69) (IgG4).

The CH2, CH3 and optional CH4 sequence can be any suitable human or human compatable sequence, e.g., as presented in FIGS. 1-42 of U.S. provisional application 60/507,349, filed 30 Sep. 2003, entirely incorporated by reference herein, corresponding to FIGS. 1-41 of PCT Appl. No. US04/19783, filed Jun. 17, 2004, entirely incorporated herein by reference, and Table 3, or as known in the art, or any combination or consensus sequence thereof, or any fusion protein thereof.

Amino acids in an EPO mimetic hinge core mimetibody or other EPO receptor agonist or specified portion or variant of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to at least one protein related activity, as specified herein or as known in the art. Sites that are critical for EPO mimetic hinge core mimetibody or other EPO receptor agonist or specified portion or variant binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

Mimetibodies or specified portions or variants of the present invention can comprise as the P portion of Formula (I), but are not limited to, at least one portion, sequence or combination selected from 3 to all the of at least one of SEQ ID NOS:1-30. Non-limiting variants that can enhance or maintain at least one of the listed activities above include, but are not limited to, any of the above polypeptides, further comprising at least one mutation corresponding to at least one substitution, insertion or deletion that does not significantly affect the suitable biological activities or functions of said EPO mimetic hinge core mimetibody or other EPO receptor agonist.

An EPO mimetic hinge core mimetibody or other EPO receptor agonist or specified portion or variant can further optionally comprise at least one functional portion of at least one polypeptide as P portion of Formula (I), at least one of 90-100% of SEQ ID NOS:1-30. An EPO mimetic hinge core mimetibody or other EPO receptor agonist can further optionally comprise an amino acid sequence for the P portion of Formula (I), selected from one or more of SEQ ID NOS:1-30.

In one embodiment, the P amino acid sequence, or portion thereof has about 90-100% identity (i.e., 90, 91, 92, 93, 94, 95, 96, 97, 98, 99, 100 or any range or value therein) to the corresponding amino acid sequence of the corresponding portion of at least one of SEQ ID NOS:1-30. Preferably, 90-100% amino acid identity (i.e., 90, 91, 92, 93, 94, 95, 96, 97, 98, 99, 100 or any range or value therein) is determined using a suitable computer algorithm, as known in the art.

Mimetibodies or specified portions or variants of the present invention can comprise any number of contiguous amino acid residues from an EPO mimetic hinge core mimetibody or other EPO receptor agonist or specified portion or variant of the present invention, wherein that number is selected from the group of integers consisting of from 10-100% of the number of contiguous residues in an EPO mimetic hinge core mimetibody or other EPO receptor agonist. Optionally, this subsequence of contiguous amino acids is at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, 5, 6, 7, 8, 9, 10, 11, 12, 13, 14, 15, 16, 17, 18, 19, 20, or more.

As those of skill will appreciate, the present invention includes at least one biologically active EPO mimetic hinge core mimetibody or other EPO receptor agonist or specified portion or variant of the present invention. Biologically active mimetibodies or specified portions or variants have a specific activity at least 20%, 30%, or 40%, and preferably at least 50%, 60%, or 70%, and most preferably at least 80%, 90%, or 95%-1000% of that of the native (non-synthetic), endogenous or related and known inserted or fused protein or specified portion or variant. Methods of assaying and quantifying measures of enzymatic activity and substrate specificity, are well known to those of skill in the art.

In another aspect, the invention relates to human mimetibodies and ligand-binding fragments, as described herein, which are modified by the covalent attachment of an organic moiety. Such modification can produce an EPO mimetic hinge core mimetibody or other EPO receptor agonist or ligand-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified mimetibodies and ligand-binding fragments of the invention can comprise one or more organic moieties that are covalently bonded, directly or indirectly, to the EPO mimetic hinge core mimetibody or other EPO receptor agonist or specified portion or variant. Each organic moiety that is bonded to an EPO mimetic hinge core mimetibody or other EPO receptor agonist or ligand-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an EPO mimetic hinge core mimetibody or other EPO receptor agonist modified by the covalent attachment of polylysine is encompassed by the invention. Hydrophilic polymers suitable for modifying mimetibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the EPO mimetic hinge core mimetibody or other EPO receptor agonist of the invention has a molecular weight of about 800 to about 150,000 Daltons as a separate molecular entity. For example, PEG₂₅₀₀, PEG₅₀₀₀, PEG₇₅₀₀, PEG₉₀₀₀, PEG₁₀₀₀₀, PEG₁₂₅₀₀, PEG₁₅₀₀₀, and PEG_{20,000}, wherein the subscript is the average molecular weight of the polymer in Daltons, can be used.

The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying mimetibodies of the invention can be saturated or can contain one or more units of unsaturation. Fatty acids that are suitable for modifying mimetibodies of the invention include, for example, n-dodecanoate (C₁₂, laurate), n-tetradecanoate (C14, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human mimetibodies and ligand-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl)carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See, for example, Thompson, et al., WO 92/16221 the entire teachings of which are incorporated herein by reference.)

The modified mimetibodies of the invention can be produced by reacting an human EPO mimetic hinge core mimetibody or other EPO receptor agonist or ligand-binding fragment with a modifying agent. For example, the organic moieties can be bonded to the EPO mimetic hinge core mimetibody or other EPO receptor agonist in a non-site specific manner by employing an amine-reactive modifying agent, for example, an NHS ester of PEG. Modified human mimetibodies or ligand-binding fragments can also be prepared by reducing disulfide bonds (e.g., intra-chain disulfide bonds) of an EPO mimetic hinge core mimetibody or other EPO receptor agonist or ligand-binding fragment. The reduced EPO mimetic hinge core mimetibody or other EPO receptor agonist or ligand-binding fragment can then be reacted with a thiol-reactive modifying agent to produce the modified EPO mimetic hinge core mimetibody or other EPO receptor agonist of the invention. Modified human mimetibodies and ligand-binding fragments comprising an organic moiety that is bonded to specific sites of an EPO mimetic hinge core mimetibody or other EPO receptor agonist or specified portion or variant of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

### EPO Mimetic Hinge Core Mimetibody or Other EPO Receptor Agonist Compositions

The present invention also provides at least one EPO mimetic hinge core mimetibody or other EPO receptor agonist or specified portion or variant composition comprising at least one, at least two, at least three, at least four, at least five, at least six or more mimetibodies or specified portions or variants thereof, as described herein and/or as known in the art that are provided in a non-naturally occurring composition, mixture or form. Such composition percentages are by weight, volume, concentration, molarity, or molality as liquid or dry solutions, mixtures, suspension, emulsions or colloids, as known in the art or as described herein.

Such compositions can comprise 0.00001-99.9999 percent by weight, volume, concentration, molarity, or molality as liquid, gas, or dry solutions, mixtures, suspension, emulsions or colloids, as known in the art or as described herein, on any range or value therein, such as but not limited to 0.00001, 0.00003, 0.00005, 0.00009, 0.0001, 0.0003, 0.0005, 0.0009, 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, 5, 6, 7, 8, 9, 10, 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9%. Such compositions of the present invention thus include but are not limited to 0.00001-100 mg/ml and/or 0.00001-100 mg/g.

The composition can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an ophthalmic, otic or nasal drug, a topical drug, a nutritional drug or the like. Such drugs are well known in the art, including formulations, indications, dosing and administration for each presented herein (see., e.g., Nursing 2001 Handbook of Drugs, 21^{st} edition, Springhouse Corp., Springhouse, Pa., 2001; Health Professional's Drug Guide 2001, ed., Shannon, Wilson, Stang, Prentice-Hall, Inc, Upper Saddle River, N.J.; Pharmacotherapy Handbook, Wells et al., ed., Appleton & Lange, Stamford, Conn., each entirely incorporated herein by reference).

EPO mimetic hinge core mimetibody or other EPO receptor agonist or specified portion or variant compositions of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the EPO mimetic hinge core mimetibody or other EPO receptor agonist composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/EPO mimetic hinge core mimetibody or other EPO receptor agonist or specified portion or variant components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

EPO mimetic hinge core mimetibody or other EPO receptor agonist compositions can also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts such as citrate.

Additionally, the EPO mimetic hinge core mimetibody or other EPO receptor agonist or specified portion or variant compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the EPO mimetic hinge core mimetibody or other EPO receptor agonist compositions according to the invention are known in the art, e.g., as listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents.

### Formulations

As noted above, the invention provides for stable formulations, which can preferably include a suitable buffer with saline or a chosen salt, as well as optional preserved solutions and formulations containing a preservative as well as multi-use preserved formulations suitable for pharmaceutical or veterinary use, comprising at least one EPO mimetic hinge core mimetibody or other EPO receptor agonist or specified portion or variant in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one EPO mimetic hinge core mimetibody or other EPO receptor agonist or specified portion or variant with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one EPO mimetic hinge core mimetibody or other EPO receptor agonist or specified portion or variant, and a second vial comprising an aqueous diluent of prescribed buffer or preservative, wherein said packaging material comprises a label that instructs a patient to reconstitute the at least one EPO mimetic hinge core mimetibody or other EPO receptor agonist or specified portion or variant in the aqueous diluent to form a solution that can be held over a period of twenty-four hours or greater.

The at least one EPO mimetic hinge core mimetibody or other EPO receptor agonist or specified portion or variant used in accordance with the present invention can be produced by recombinant means, including from mammalian cell or transgenic preparations, or can be purified from other biological sources, as described herein or as known in the art.

The range of amounts of at least one EPO mimetic hinge core mimetibody or other EPO receptor agonist or specified portion or variant in the product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g. isotonicity agents, buffers, antioxidants, preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably the formulations of the present invention have pH between about 6.8 and about 7.8. Preferred buffers include phosphate buffers, most preferably sodium phosphate, particularly phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one EPO mimetic hinge core mimetibody or other EPO receptor agonist or specified portion or variant and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one EPO mimetic hinge core mimetibody or other EPO receptor agonist or specified portion or variant and preservative in an aqueous diluent is carried out using conventional dissolution and mixing procedures. To prepare a suitable formulation, for example, a measured amount of at least one EPO mimetic hinge core mimetibody or other EPO receptor agonist or specified portion or variant in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that may be optimized for the concentration and means of administration used.

The claimed formulations can be provided to patients as clear solutions or as dual vials comprising a vial of lyophilized at least one EPO mimetic hinge core mimetibody or other EPO receptor agonist or specified portion or variant that is reconstituted with a second vial containing water, a preservative and/or excipients, preferably a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period of immediately to twenty-four hours or greater. Accordingly, the presently claimed articles of manufacture offer significant advantages to the patient. Formulations of the invention can optionally be safely stored at temperatures of from about 2 to about 40° C. and retain the biologically activity of the protein for extended periods of time, thus, allowing a package label indicating that the solution can be held and/or used over a period of 6, 12, 18, 24, 36, 48, 72, or 96 hours or greater. If preserved diluent is used, such label can include use up to at least one of 1-12 months, one-half, one and a half, and/or two years.

The solutions of at least one EPO mimetic hinge core mimetibody or other EPO receptor agonist or specified portion or variant in the invention can be prepared by a process that comprises mixing at least one EPO mimetic hinge core mimetibody or other EPO receptor agonist or specified portion or variant in an aqueous diluent. Mixing is carried out using conventional dissolution and mixing procedures. To prepare a suitable diluent, for example, a measured amount of at least one EPO mimetic hinge core mimetibody or other EPO receptor agonist or specified portion or variant in water or buffer is combined in quantities sufficient to provide the protein and optionally a preservative or buffer at the desired concentrations. Variations of this process would be recognized by one of ordinary skill in the art. For example, the order the components are added, whether additional additives are used, the temperature and pH at which the formulation is prepared, are all factors that may be optimized for the concentration and means of administration used.

The claimed products can be provided to patients as clear solutions or as dual vials comprising a vial of lyophilized at least one EPO mimetic hinge core mimetibody or other EPO receptor agonist or specified portion or variant that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one EPO mimetic hinge core mimetibody or other EPO receptor agonist or specified portion or variant that is reconstituted with a second vial containing the aqueous diluent. The clear solution in this case can be up to one liter or even larger in size, providing a large reservoir from which smaller portions of the at least one EPO mimetic hinge core mimetibody or other EPO receptor agonist or specified portion or variant solution can be retrieved one or multiple times for transfer into smaller vials and provided by the pharmacy or clinic to their customers and/or patients.

Recognized devices comprising these single vial systems include single or dual vial pen-injector devices for delivery of a drug solution for use in the present invention, as well as optionally further comprising a dual vial system include those pen-injector systems for reconstituting a lyophilized drug in a cartridge for delivery of the drug solution, as well known in the art. such as those disclosed in U.S. Pat. Nos. 6,203,530, 5,026,349, 5,567,160, 5,954,738, 5,879,327, 5,599,302, 6,015,438, 6,461,333, 6,517,517, 6,077,247, 6,099,504, 6,428,528, 6,387,078, 5,868,711, 5,960,797, 5,176,643, 5,271,744, 5,405,362, 5,451,210, 5,137,516, 5,176,643, 5,300,030, 5,295,965, 5,425,715, 5,478,316, 5,575,777, 5,599,309, 5,681,291, 5,709,662, 5,779,677, 5,913,843, 5,843,036, 5,957,897, 6,428,528, 6,086,562, 6,099,503, 6,221,044, 6,270,479, 6,258,068, 6,391,003, 6,280,421, 6,045,534, 6,371,939, 6,090,078, 5,267,963, 5,487,732, 5,425,715, 6,575,939, 6,565,540, 6,159,181. 6,179,812, 6,544,234, 6,572,581, 6,676,630, 6,083,197, 6,203,530, 6,270,479, 6,371,939, 5,575,777, 6,090,078, 6,743,199, 6,589,210, 6,689,093, 6,783,509, 6,645,170, 6,641,554, 6,796,967, 6,645,181, 6,641,565, 6,575,939, 6,569,115, 6,673,049, 6,641,560, 6,569,124, 6,699,220, 6,638,256, 6,607,508, 6,607,510, 6,776,777, 6,767,336, 6,595,962, 6,740,062, 6,565,553, 6,746,429, 6,569,123, 6,595,957, 6,770,056, which are each entirely incorporated herein by reference.

The products presently claimed include packaging material. The packaging material provides, in addition to the information required by the regulatory agencies, the conditions under which the product can be used. The packaging material of the present invention provides instructions to the patient to reconstitute the at least one EPO mimetic hinge core mimetibody or other EPO receptor agonist or specified portion or variant in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one EPO mimetic hinge core mimetibody or other EPO receptor agonist or specified portion or variant and a selected buffer, preferably a phosphate buffer containing saline or a chosen salt. Mixing the at least one EPO mimetic hinge core mimetibody or other EPO receptor agonist or specified portion or variant and buffer in an aqueous diluent is carried out using conventional dissolution and mixing procedures. To prepare a suitable formulation, for example, a measured amount of at least one EPO mimetic hinge core mimetibody or other EPO receptor agonist or specified portion or variant in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized at least one EPO mimetic hinge core mimetibody or other EPO receptor agonist or specified portion or variant that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

At least one EPO mimetic hinge core mimetibody or other EPO receptor agonist or specified portion or variant in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

### Therapeutic Applications

The present invention for mimetibodies also provides a method for modulating or treating glucose intolerance related disorders and/or renal disease related anemia, in a cell, tissue, organ, animal, or patient.

The present invention also provides a method for modulating or treating a glucose intolerance related disorders and/or renal disease related anemia or blood cell related condition, in a cell, tissue, organ, animal, or patient, wherein said anemia or blood cell related condition is associated with at least one including, but not limited to, at least one of immune related disease, cardiovascular disease, infectious, malignant and/or neurologic disease. Such a method can optionally comprise administering an effective amount of at least one composition or pharmaceutical composition comprising at least one EPO mimetic hinge core mimetibody or other EPO receptor agonist or specified portion or variant to a cell, tissue, organ, animal or patient in need of such modulation, treatment or therapy.

Any method of the present invention can comprise administering an effective amount of a composition or pharmaceutical composition comprising at least one EPO mimetic hinge core mimetibody or other EPO receptor agonist or specified portion or variant to a cell, tissue, organ, animal or patient in need of such modulation, treatment or therapy. Such a method can optionally further comprise co-administration or combination therapy for treating such immune diseases, wherein the administering of said at least one EPO mimetic hinge core mimetibody or other EPO receptor agonist, specified portion or variant thereof, further comprises administering, before concurrently, and/or after, at least one selected from at least one TNF antagonist (e.g., but not limited to a TNF antibody or fragment, a soluble TNF receptor or fragment, fusion proteins thereof, or a small molecule TNF antagonist), an antirheumatic, a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a fluoroquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

As used herein, a “tumor necrosis factor antibody,” “TNF antibody,” “TNFα antibody,” or fragment and the like decreases, blocks, inhibits, abrogates or interferes with TNFα activity in vitro, in situ and/or preferably in vivo. For example, a suitable TNF human antibody of the present invention can bind TNFα and includes anti-TNF antibodies, antigen-binding fragments thereof, and specified mutants or domains thereof that bind specifically to TNFα. A suitable TNF antibody or fragment can also decrease block, abrogate, interfere, prevent and/or inhibit TNF RNA, DNA or protein synthesis, TNF release, TNF receptor signaling, membrane TNF cleavage, TNF activity, TNF production and/or synthesis.

Typically, treatment of pathologic conditions is effected by administering an effective amount or dosage of at least one EPO mimetic hinge core mimetibody or other EPO receptor agonist composition that total, on average, a range from at least about 0.001 to 500 milligrams of at least one EPO mimetic hinge core mimetibody or other EPO receptor agonist or specified portion or variant/kilogram of patient per dose, and preferably from at least about 0.01 to 100 milligrams EPO mimetic hinge core mimetibody or other EPO receptor agonist or specified portion or variant/kilogram of patient per single or multiple administration, depending upon the specific activity of contained in the composition. Alternatively, the effective serum concentration can comprise 0.001-5000 μg/ml serum concentration per single or multiple administration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.01, 0.02, 0.03, 0.04, 0.05. 0.06, 0.07, 0.08, 009, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, and/or 30 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably 0.1 to 10 milligrams per kilogram per administration or in sustained release form is effective to obtain desired results.

As a non-limiting example, treatment of humans or animals can be provided as a one-time or periodic dosage of at least one EPO mimetic hinge core mimetibody or other EPO receptor agonist or specified portion or variant of the present invention 0.01 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20, or any combination thereof, using single, infusion or repeated doses.

Dosage forms (composition) suitable for internal administration generally contain from about 0.0001 milligram to about 500 milligrams of active ingredient per unit or container. In these pharmaceutical compositions the active ingredient will ordinarily be present in an amount of about 0.5-95% by weight based on the total weight of the composition.

For parenteral administration, the EPO mimetic hinge core mimetibody or other EPO receptor agonist or specified portion or variant can be formulated as a solution, suspension, emulsion or lyophilized powder in association, or separately provided, with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Therapeutic Administration

Many known and developed modes of can be used according to the present invention for administering pharmaceutically effective amounts of at least one EPO mimetic hinge core mimetibody or other EPO receptor agonist or specified portion or variant according to the present invention. While pulmonary administration is used in the following description, other modes of administration can be used according to the present invention with suitable results.

An EPO mimetic hinge core mimetibody or other EPO receptor agonist of the present invention can be delivered in a carrier, as a solution, emulsion, colloid, or suspension, or as a powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent such as aqueous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

### Alternative Delivery

The invention further relates to the administration of at least one EPO mimetic hinge core mimetibody or other EPO receptor agonist or specified portion or variant by parenteral, subcutaneous, intramuscular, intravenous, bolus, vaginal, rectal, buccal, sublingual, intranasal, or transdermal means. EPO mimetic hinge core mimetibody or other EPO receptor agonist or specified portion or variant compositions can be prepared for use for parenteral (subcutaneous, intramuscular or intravenous) administration particularly in the form of liquid solutions or suspensions; for use in vaginal or rectal administration particularly in semisolid forms such as creams and suppositories; for buccal, or sublingual administration particularly in the form of tablets or capsules; or intranasally particularly in the form of powders, nasal drops or aerosols or certain agents; or transdermally particularly in the form of a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement”; Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways such as electroporation, or to increase the mobility of charged drugs through the skin such as iontophoresis, or application of ultrasound such as sonophoresis (U.S. Pat. Nos. 4,309,989 and 4,767,402) (the above publications and patents being entirely incorporated herein by reference).

### Pulmonary/Nasal Administration

For pulmonary administration, preferably at least one EPO mimetic hinge core mimetibody or other EPO receptor agonist or specified portion or variant composition is delivered in a particle size effective for reaching the lower airways of the lung or sinuses. According to the invention, at least one EPO mimetic hinge core mimetibody or other EPO receptor agonist or specified portion or variant can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of EPO mimetic hinge core mimetibody or other EPO receptor agonist or specified portion or variants are also known in the art. All such devices can use of formulations suitable for the administration for the dispensing of EPO mimetic hinge core mimetibody or other EPO receptor agonist or specified portion or variant in an aerosol. Such aerosols can be comprised of either solutions (both aqueous and non aqueous) or solid particles. Metered dose inhalers like the Ventolin® metered dose inhaler, typically use a propellent gas and require actuation during inspiration (See, e.g., WO 94/16970, WO 98/35888). Dry powder inhalers like Turbuhaler™ (Astra), Rotahaler® (Glaxo), Diskus® (Glaxo), Spiros™ inhaler (Dura), devices marketed by Inhale Therapeutics, and the Spinhaler® powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent® nebulizer (Mallinckrodt), and the Acorn II® nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention. Preferably, a composition comprising at least one EPO mimetic hinge core mimetibody or other EPO receptor agonist or specified portion or variant is delivered by a dry powder inhaler or a sprayer. There are a several desirable features of an inhalation device for administering at least one EPO mimetic hinge core mimetibody or other EPO receptor agonist or specified portion or variant of the present invention. For example, delivery by the inhalation device is advantageously reliable, reproducible, and accurate. The inhalation device can optionally deliver small dry particles, e.g. less than about 10 μm, preferably about 1-5 μm, for good respirability.

### Administration of EPO Mimetic Hinge Core Mimetibody or Other EPO Receptor Agonist or Specified Portion or Variant Compositions as a Spray

A spray including EPO mimetic hinge core mimetibody or other EPO receptor agonist or specified portion or variant composition protein can be produced by forcing a suspension or solution of at least one EPO mimetic hinge core mimetibody or other EPO receptor agonist or specified portion or variant through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one EPO mimetic hinge core mimetibody or other EPO receptor agonist or specified portion or variant composition protein delivered by a sprayer have a particle size less than about 10 μm, preferably in the range of about 1 μm to about 5 μm, and most preferably about 2 μm to about 3 μml.

Formulations of at least one EPO mimetic hinge core mimetibody or other EPO receptor agonist or specified portion or variant composition protein suitable for use with a sprayer typically include EPO mimetic hinge core mimetibody or other EPO receptor agonist or specified portion or variant composition protein in an aqueous solution at a concentration of about 1 mg to about 20 mg of at least one EPO mimetic hinge core mimetibody or other EPO receptor agonist or specified portion or variant composition protein per ml of solution. The formulation can include agents such as an excipient, a buffer, an isotonicity agent, a preservative, a surfactant, and, preferably, zinc. The formulation can also include an excipient or agent for stabilization of the EPO mimetic hinge core mimetibody or other EPO receptor agonist or specified portion or variant composition protein, such as a buffer, a reducing agent, a bulk protein, or a carbohydrate. Bulk proteins useful in formulating EPO mimetic hinge core mimetibody or other EPO receptor agonist or specified portion or variant composition proteins include albumin, protamine, or the like. Typical carbohydrates useful in formulating EPO mimetic hinge core mimetibody or other EPO receptor agonist or specified portion or variant composition proteins include sucrose, mannitol, lactose, trehalose, glucose, or the like. The EPO mimetic hinge core mimetibody or other EPO receptor agonist or specified portion or variant composition protein formulation can also include a surfactant, which can reduce or prevent surface-induced aggregation of the EPO mimetic hinge core mimetibody or other EPO receptor agonist or specified portion or variant composition protein caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of a protein such as mimetibodies, or specified portions or variants, can also be included in the formulation.

### Administration of EPO Mimetic Hinge Core Mimetibody or Other EPO Receptor Agonist or Specified Portion or Variant Compositions by a Nebulizer

EPO mimetic hinge core mimetibody or other EPO receptor agonist or specified portion or variant composition protein can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of EPO mimetic hinge core mimetibody or other EPO receptor agonist or specified portion or variant composition protein through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of EPO mimetic hinge core mimetibody or other EPO receptor agonist or specified portion or variant composition protein either directly or through a coupling fluid, creating an aerosol including the EPO mimetic hinge core mimetibody or other EPO receptor agonist or specified portion or variant composition protein. Advantageously, particles of EPO mimetic hinge core mimetibody or other EPO receptor agonist or specified portion or variant composition protein delivered by a nebulizer have a particle size less than about 10 μm, preferably in the range of about 1 μm to about 5 μm, and most preferably about 2 μm to about 3 μm.

Formulations of at least one EPO mimetic hinge core mimetibody or other EPO receptor agonist or specified portion or variant suitable for use with a nebulizer, either jet or ultrasonic, typically include EPO mimetic hinge core mimetibody or other EPO receptor agonist or specified portion or variant composition protein in an aqueous solution at a concentration of about 1 mg to about 20 mg of at least one EPO mimetic hinge core mimetibody or other EPO receptor agonist or specified portion or variant protein per ml of solution. The formulation can include agents such as an excipient, a buffer, an isotonicity agent, a preservative, a surfactant, and, preferably, zinc. The formulation can also include an excipient or agent for stabilization of the at least one EPO mimetic hinge core mimetibody or other EPO receptor agonist or specified portion or variant composition protein, such as a buffer, a reducing agent, a bulk protein, or a carbohydrate. Bulk proteins useful in formulating at least one EPO mimetic hinge core mimetibody or other EPO receptor agonist or specified portion or variant composition proteins include albumin, protamine, or the like. Typical carbohydrates useful in formulating at least one EPO mimetic hinge core mimetibody or other EPO receptor agonist or specified portion or variant include sucrose, mannitol, lactose, trehalose, glucose, or the like. The at least one EPO mimetic hinge core mimetibody or other EPO receptor agonist or specified portion or variant formulation can also include a surfactant, which can reduce or prevent surface-induced aggregation of the at least one EPO mimetic hinge core mimetibody or other EPO receptor agonist or specified portion or variant caused by atomization of the solution in forming an aerosol. Various conventional surfactants can be employed, such as polyoxyethylene fatty acid esters and alcohols, and polyoxyethylene sorbital fatty acid esters. Amounts will generally range between 0.001 and 4% by weight of the formulation. Especially preferred surfactants for purposes of this invention are polyoxyethylene sorbitan mono-oleate, polysorbate 80, polysorbate 20, or the like. Additional agents known in the art for formulation of a protein such as at least one EPO mimetic hinge core mimetibody or other EPO receptor agonist or specified portion or variant protein can also be included in the formulation.

### Administration of EPO Mimetic Hinge Core Mimetibody or Other EPO Receptor Agonist or Specified Portion or Variant Compositions by a Metered Dose Inhaler

In a metered dose inhaler (MDI), a propellant, at least one EPO mimetic hinge core mimetibody or other EPO receptor agonist or specified portion or variant, and any excipients or other additives are contained in a canister as a mixture including a liquefied compressed gas. Actuation of the metering valve releases the mixture as an aerosol, preferably containing particles in the size range of less than about 10 μm, preferably about 1 μm to about 5 μm, and most preferably about 2 μm to about 3 μm. The desired aerosol particle size can be obtained by employing a formulation of EPO mimetic hinge core mimetibody or other EPO receptor agonist or specified portion or variant composition protein produced by various methods known to those of skill in the art, including jet-milling, spray drying, critical point condensation, or the like. Preferred metered dose inhalers include those manufactured by 3M or Glaxo and employing a hydrofluorocarbon propellant.

Formulations of at least one EPO mimetic hinge core mimetibody or other EPO receptor agonist or specified portion or variant for use with a metered-dose inhaler device will generally include a finely divided powder containing at least one EPO mimetic hinge core mimetibody or other EPO receptor agonist or specified portion or variant as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluoroalkane-134a), HFA-227 (hydrofluoroalkane-227), or the like. Preferably the propellant is a hydrofluorocarbon. The surfactant can be chosen to stabilize the at least one EPO mimetic hinge core mimetibody or other EPO receptor agonist or specified portion or variant as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol. Additional agents known in the art for formulation of a protein such as protein can also be included in the formulation.

One of ordinary skill in the art will recognize that the methods of the current invention can be achieved by pulmonary administration of at least one EPO mimetic hinge core mimetibody or other EPO receptor agonist or specified portion or variant compositions via devices not described herein.

### Mucosal Formulations and Administration

For absorption through mucosal surfaces, compositions and methods of administering at least one EPO mimetic hinge core mimetibody or other EPO receptor agonist or specified portion or variant include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g. suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration excipients include sugars, calcium stearate, magnesium stearate, pregelinatined starch, and the like (U.S. Pat. No. 5,849,695).

### Oral Formulations and Administration

Formulations for oral rely on the co-administration of adjuvants (e.g., resorcinols and nonionic surfactants such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant such as magnesium stearate, paraben, preserving agent such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations may contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. No. 5,879,681 and U.S. Pat. No. 5,5,871,753 are used to deliver biologically active agents orally are known in the art.

### Transdermal Formulations and Administration

For transdermal administration, the at least one EPO mimetic hinge core mimetibody or other EPO receptor agonist or specified portion or variant is encapsulated in a delivery device such as a liposome or polymeric nanoparticles, microparticle, microcapsule, or microspheres (referred to collectively as microparticles unless otherwise stated). A number of suitable devices are known, including microparticles made of synthetic polymers such as polyhydroxy acids such as polylactic acid, polyglycolic acid and copolymers thereof, polyorthoesters, polyanhydrides, and polyphosphazenes, and natural polymers such as collagen, polyamino acids, albumin and other proteins, alginate and other polysaccharides, and combinations thereof (U.S. Pat. No. 5,814,599).

### Prolonged Administration and Formulations

It can be sometimes desirable to deliver the compounds of the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b) e.g. a zinc tannate salt. Additionally, the compounds of the present invention or, preferably, a relatively insoluble salt such as those just described, can be formulated in a gel, for example, an aluminum monostearate gel with, e.g. sesame oil, suitable for injection. Particularly preferred salts are zinc salts, zinc tannate salts, pamoate salts, and the like. Another type of slow release depot formulation for injection would contain the compound or salt dispersed for encapsulated in a slow degrading, non-toxic, non-antigenic polymer such as a polylactic acid/polyglycolic acid polymer for example as described in U.S. Pat. No. 3,773,919. The compounds or, preferably, relatively insoluble salts such as those described above can also be formulated in cholesterol matrix silastic pellets, particularly for use in animals. Additional slow release, depot or implant formulations, e.g. gas or liquid liposomes are known in the literature (U.S. Pat. No. 5,770,222 and “Sustained and Controlled Release Drug Delivery Systems”, J. R. Robinson ed., Marcel Dekker, Inc., N.Y., 1978).

Having generally described the invention, the same will be more readily understood by reference to the following examples, which are provided by way of illustration and are not intended as limiting.

### Example 1

### Cloning and Expression of an EPO Mimetic Hinge Core Mimetibody or Other EPO Receptor Agonist in Mammalian Cells

A typical mammalian expression vector contains at least one promoter element, which mediates the initiation of transcription of mRNA, the EPO mimetic hinge core mimetibody or other EPO receptor agonist or specified portion or variant coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded EPO mimetic hinge core mimetibody or other EPO receptor agonist or specified portion or variant. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of EPO mimetic hinge core mimetibody or other EPO receptor agonist or specified portion or variants.

The expression vectors pC1 and pC4 contain the strong promoter (LTR) of the Rous Sarcoma Virus (Cullen, et al., Molec. Cell. Biol. 5:438-447 (1985)) plus a fragment of the CMV-enhancer (Boshart, et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### Cloning and Expression in CHO Cells

The vector pC4 is used for the expression of EPO mimetic hinge core mimetibody or other EPO receptor agonist or specified portion or variant. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (e.g., alpha minus MEM, Life Technologies, Gaithersburg, Md.) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained that contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the EPO in a regulated way in mammalian cells (M. Gossen, and H. Bujard, Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete EPO mimetic hinge core mimetibody or other EPO receptor agonist or specified portion or variant is used, corresponding to HC and LC variable regions of an EPO mimetic hinge core mimetibody or other EPO receptor agonist of the present invention, according to known method steps. Isolated nucleic acid encoding a suitable human constant region (i.e., HC and LC regions) is also used in this construct.

The isolated variable and constant region encoding DNA and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained that grow at a concentration of 100-200 mM. Expression of the desired gene product is analyzed, for instance, by SDS-PAGE and Western blot or by reverse phase HPLC analysis.

### Example 2

### Non-Limiting Example of an EPO Mimetic Hinge Core Mimetibody of the Invention

Background: EMP-1 (EPO mimetic peptide-1) is a 20 amino acid peptide with no sequence homology to human erythropoietin (HuEPO), but with the ability (as a dimer) to activate the EPO receptor (Wrighton et al, 1996, Science, vol. 273, 458-463). However, its relatively low activity (10,000 to 100,000 fold less than HuEPO) and short half-life (ex-vivo half-life of 8 hours in 50% serum, in vivo half-life unknown), compromise its utility as a therapeutic. Therefore, a way was needed to confer upon the peptide a longer half-life, without disturbing, and possibly improving its potency. To this end, several attempts have been made to increase the activity of EMP-1 by stabilizing the dimerization of the peptide or by incorporating the peptide into larger structures to increase half-life. Wrighten et al. (1997, Nature Biotechnology, vol. 15, 1261-65) combined biotin labeled EMP-1 with streptavidin to stabilize dimerization. They saw a 100 fold increase in activity in an in vitro cell proliferation assay. They also used anti-biotin antibodies to stabilize the peptide dimer, however only a 10-fold increase in activity was seen. The same authors prepared a chemically defined dimeric form of EMP-1. In this case an 100-fold increase in activity was seen in vivo. Another group sought to improve the activity of EMP-1 through covalent linkage to polyethylene glycol (PEG) (Johnson et al., 1997, Chem. & Bio., vol. 4(12), 939-50). They reported an increase in potency of up to 1000 fold, however the construct was found to be immunogenic in mice (the antibodies were directed to the peptide) (Dana Johnson, Personal communications). Kuai et al. (2000, J. Peptide Res., vol. 56, 59-62) inserted the EMP-1 peptide into the sequence of plasminogen activator inhibitor-1, (PAI-1). It was thought that the insertion of EMP-1 into this scaffold would both stabilize dimerization and increase half-life. In an in vivo assay the potency of this construct was seen to be significantly higher, such as more than 2500 fold higher than EMP-1 alone. It should be noted that different in vitro assays and in vivo models were used in these studies and the reported potencies may not be comparable to each other or to results presented herein.

### Example 3

### EPO Mimetic Hinge Core Mimetibody of the Present Invention

A specific, non-limiting, example of this invention is the EMP-hinge core mimetibody construct where V is the first several N-terminal amino acids of a naturally occurring HC or LC antibody, P is a single copy of the bioactive EMP-1 peptide and L is a tandem repeat of either Gly-Ser or Gly-Gly-Gly-Ser flexible linker, H is a hinge core region and CH2 & CH3 are of the IgG1 or IgG4 isotype subclass. It is thought that this structure will constrain the EMP-1 peptide, but allow sufficient flexibility such that the dimerization of the peptides as part of the assembled homodimer is stabilized. In support of this, the activity of EMP-hinge core mimetibody in an in vitro cell proliferation assay is more than 500 fold greater than the EMP-1 peptide and only substantially similar to recombinant HuEPO (rHuEPO). In addition, it is expected that the half-life of this construct will be many times that of rHuEPO or the EMP-1 peptide alone and similar to that of an IgG. Consistently, normal mice treated with EMP-hinge core mimetibody attain a significantly higher maximal hematocrit compared to mice treated with rHuEPO, when equal activity units are given, and elevated levels are maintained for a longer period. This construct is efficiently secreted from cells and appears to be properly folded; overcoming problems associated with 1^{st} generation mimetibodies.

In addition to the basic structure described above, variants with potentially favorable biological characteristics are described. These include constructs that may have a decreased tendency to self-associate, reduced immune effector functions or decreased immunogenicity. Other modifications that confer desired characteristics such as improved conformation of the biologically active peptide, and transfer across the blood-brain barrier are envisioned. The proposed variants and modifications may be combined in any fashion to yield constructs with desired activities.

Using recombinant DNA methods, the EMP-1 peptide was inserted into an intermediate vector between an immunoglobulin signal peptide and a human J sequence. This was done using complementary synthetic oligonucleotides with ends compatible with the restriction sites present in the vector These oligonucleotides comprised coding sequences for the signal peptidase consensus site (QIQ), the EMP-1 peptide (SEQ ID NO:2), and a flexible linker composed of either GS or GGGS. A restriction fragment containing the above-mentioned functional elements was then transferred into an expression vector. This vector contained the anti-CD4 immunoglobulin promoter and enhancer, and the coding sequence for a human IgG1 hinge core sequence, and a portion of an IgG1 hinge core region, CPPCP (109-113 of SEQ ID NO:66, as shown in FIG. 36C), an HC constant region 2 (CH2) and constant region 3 (CH3) as well as the necessary elements for plasmid replication and selection in bacteria and selection for stable expressers in mammalian cells.

This plasmid was linearized and introduced into the NSO mouse myeloma cell line via electroporation. Resistant cells were selected and high expressers of EMP-hinge core mimetibody were identified by ELISA assay of culture supernatants. Purification of the construct from cell culture supernatants was accomplished by standard proteinA affinity chromatography. Passage of the purified product through SDS-containing polyacrylamide gels under both denaturing and reducing conditions confirmed the expected size of the purified product. The identity of the purified protein was further confirmed by mass spectrometry and N-terminal sequencing.

The amino acid sequences of EMP-hinge core mimetibodies are shown below. Functional domains are annotated above the peptide coding sequence. The three amino acid signal peptide consensus sequence corresponds to the first three amino acids of a naturally occurring immunoglobulin. These amino acids are thought to contribute to the efficient removal of the signal peptide by signal peptidase in the endoplasmic reticulum. This sequence is immediately followed by the EMP-1 coding sequence. The two C-terminal amino acids of the EMP-1 sequence combined with the next six amino acids form a flexible linker characterized by the Gly-Gly-Gly-Ser repeat. A human joining (J) region sequence follows. It is thought that the J sequence will provide even more flexibility to allow the EMP-1 dimmer to assume the proper conformation, and allow the dimmer to protrude from the globular structure of the immunoglobulin and penetrate into the cleft between two EPO receptors. The HC hinge region is also included in the construct immediately following the J region. There are three cysteines in the IgG1 hinge region (highlighted). The first would normally pair to the immunoglobulin light chain (LC) and the second two participate in interchain bonds between two HCs. The remainder of the sequence is composed of the CH2 & CH3 regions, which constitute the bulk of the protein. One of the reasons that immunoglobulins are believed to have a long serum half-life is their ability to bind the FcRn that extends the serum half-life by returning pinocytosed immunoglobulin back to the extracellular space. The binding site of the FcRn overlaps the junction of the CH2 and CH3 regions (Sheilds et al, 2001, J. Biol. Chem., vol. 276 (9), 6591-6604).

The peptide sequence of EMP-hinge core mimetibody showing important functional domains.

| | V EMP-1 Peptide Linker Hinge IgG1 CH2 | |
|---|---|---|
| 1 | QIQGGTYSCHFGPLTWVCKPQGG GS CPPCP APELLGGP | |
| | IgG1 CH2 ----------------------------------------------------- | |
| 61 | SVFLFPPKPKDTLMISRTPEVTCVVVDVSHEDPEVKFNWYVDGVEVHNAKTKPREEQYNS | |
| | ~~~~~~~~~~~~~~~~~~~~~~~~~~~~~~~~~~~~~~~~~~ IgG1 CH3 | |
| 122 | TYRVVSVLTVLHQDWLNGKEYKCKVSNKALPAPIEKTISKAKGQPREPQVYTLPPSRDEL | |
| | IgG1 CH3 | |
| 183 | TKNQVSLTCLVKGFYPSDIAVEWESNGQPENNYKTTPPVLDSDGSFFLYSKLTVDKSRWQ | |
| | IgG1 CH3 | |
| 241 | QGNVFSCSVMHEALHNHYTQKSLSLSPGK | (SEQ ID NO: 82) |
| | V EMP-1 Peptide Linker Hinge IgG1 CH2 | |
| 1 | QIQGGTYSCHFGPLTWVCKPQGG GGGS CPPCP APELLGGP | |
| | IgG1 CH2 ----------------------------------------------------- | |
| 61 | SVFLFPPKPKDTLMISRTPEVTCVVVDVSHEDPEVKFNWYVDGVEVHNAKTKPREEQYNS | |
| | ~~~~~~~~~~~~~~~~~~~~~~~~~~~~~~~~~~~~~~~~~~ IgG1 CH3 | |
| 122 | TYRVVSVLTVLHQDWLNGKEYKCKVSNKALPAPIEKTISKAKGQPREPQVYTLPPSRDEL | |
| | IgG1 CH3 | |
| 183 | TKNQVSLTCLVKGFYPSDIAVEWESNGQPENNYKTTPPVLDSDGSFFLYSKLTVDKSRWQ | |
| | IgG1 CH3 | |
| 241 | QGNVFSCSVMHEALHNHYTQKSLSLSPGK | (SEQ ID NO: 83) |
| | V EMP-1 Peptide Linker Hinge IgG1 CH2 | |
| 1 | QIQGGTYSCHFGPLTWVCKPQGG GSGGGS CPPCP APELLGGP | |
| | IgG1 CH2 ----------------------------------------------------- | |
| 61 | SVFLFPPKPKDTLMISRTPEVTCVVVDVSHEDPEVKFNWYVDGVEVHNAKTKPREEQYNS | |
| | ~~~~~~~~~~~~~~~~~~~~~~~~~~~~~~~~~~~~~~~~~~ IgG1 CH3 | |
| 122 | TYRVVSVLTVLHQDWLNGKEYKCKVSNKALPAPIEKTISKAKGQPREPQVYTLPPSRDEL | |
| | IgG1 CH3 | |
| 183 | TKNQVSLTCLVKGFYPSDIAVEWESNGQPENNYKTTPPVLDSDGSFFLYSKLTVDKSRWQ | |
| | IgG1 CH3 | |
| 241 | QGNVFSCSVMHEALHNHYTQKSLSLSPGK | (SEQ ID NO: 84) |
| | V EMP-1 Peptide Linker Hinge IgG1 CH2 | |
| 1 | QIQGGTYSCHFGPLTWVCKPQGG GS CPPCP APEAAGGP | |
| | IgG1 CH2 ----------------------------------------------------- | |
| 61 | SVFLFPPKPKDTLMISRTPEVTCVVVDVSHEDPEVKFNWYVDGVEVHNAKTKPREEQYNS | |
| | ~~~~~~~~~~~~~~~~~~~~~~~~~~~~~~~~~~~~~~~~~~ IgG1 CH3 | |
| 122 | TYRVVSVLTVLHQDWLNGKEYKCKVSNKALPAPIEKTISKAKGQPREPQVYTLPPSRDEL | |
| | IgG1 CH3 | |
| 183 | TKNQVSLTCLVKGFYPSDIAVEWESNGQPENNYKTTPPVLDSDGSFFLYSKLTVDKSRWQ | |
| | IgG1 CH3 | |
| 241 | QGNVFSCSVMHEALHNHYTQKSLSLSPGK | (SEQ ID NO: 85) |
| | V EMP-1 Peptide Linker Hinge IgG1 CH2 | |
| 1 | QIQGGTYSCHFGPLTWVCKPQGG GGGS CPPCP APEAAGGP | |
| | IgG1 CH2 ----------------------------------------------------- | |
| 61 | SVFLFPPKPKDTLMISRTPEVTCVVVDVSHEDPEVKFNWYVDGVEVHNAKTKPREEQYNS | |
| | ~~~~~~~~~~~~~~~~~~~~~~~~~~~~~~~~~~~~~~~~~~ IgG1 CH3 | |
| 122 | TYRVVSVLTVLHQDWLNGKEYKCKVSNKALPAPIEKTISKAKGQPREPQVYTLPPSRDEL | |
| | IgG1 CH3 | |
| 183 | TKNQVSLTCLVKGFYPSDIAVEWESNGQPENNYKTTPPVLDSDGSFFLYSKLTVDKSRWQ | |
| | IgG1 CH3 | |
| 241 | QGNVFSCSVMHEALHNHYTQKSLSLSPGK | (SEQ ID NO: 86) |
| | V EMP-1 Peptide Linker Hinge IgG4 CH2 | |
| 1 | QIQGGTYSCHFGPLTWVCKPQGG GS CPPCP APEFLGGP | |
| | IgG 4 CH2 --------------------------------------- | |
| 61 | SVFLFPPKPKDTLMISRTPEVTCVVVDVSQEDPEVQFNWYVDGVEVHNAKTKPREEQFNS | |
| | ~~~~~~~~~~~~~~~~~~~~~~~~~~~~~~~~~~~~~~~~~~ IgG4 CH3 | |
| 121 | TYRVVSVLTVLHQDWLNGKEYKCKVSNKGLPSSIEKTISKAKGQPREPQVYTLPPSQEEM | |
| | IgG4 CH3 | |
| 183 | TKNQVSLTCLVKGFYPSDIAVEWESNGQPENNYKTTPPVLDSDGSFFLYSRLTVDKSRWQ | |
| | IgG4 CH3 | |
| 241 | EGNVFSCSVMHEALHNHYTQKSLSLSLGK | (SEQ ID NO: 87) |
| | V EMP-1 Peptide Linker Hinge IgG4 CH2 | |
| 1 | QIQGGTYSCHFGPLTWVCKPQGG GS CPPCP APEAAGGP | |
| | IgG 4 CH2 --------------------------------------- | |
| 61 | SVFLFPPKPKDTLMISRTPEVTCVVVDVSQEDPEVQFNWYVDGVEVHNAKTKPREEQFNS | |
| | ~~~~~~~~~~~~~~~~~~~~~~~~~~~~~~~~~~~~~~~~~~ IgG4 CH3 | |
| 121 | TYRVVSVLTVLHQDWLNGKEYKCKVSNKGLPSSIEKTISKAKGQPREPQVYTLPPSQEEM | |
| | IgG4 CH3 | |
| 183 | TKNQVSLTCLVKGFYPSDIAVEWESNGQPENNYKTTPPVLDSDGSFFLYSRLTVDKSRWQ | |
| | IgG4 CH3 | |
| 241 | EGNVFSCSVMHEALHNHYTQKSLSLSLGK | (SEQ ID NO: 88) |
| | V EMP-1 Peptide Linker Hinge IgG4 CH2 | |
| 1 | QIQGGTYSCHFGPLTWVCKPQGG GGGS CPPCP APEAAGGP | |
| | IgG 4 CH2 --------------------------------------- | |
| 61 | SVFLFPPKPKDTLMISRTPEVTCVVVDVSQEDPEVQFNWYVDGVEVHNAKTKPREEQFNS | |
| | ~~~~~~~~~~~~~~~~~~~~~~~~~~~~~~~~~~~~~~~~~~ IgG4 CH3 | |
| 121 | TYRVVSVLTVLHQDWLNGKEYKCKVSNKGLPSSIEKTISKAKGQPREPQVYTLPPSQEEM | |
| | IgG4 CH3 | |
| 183 | TKNQVSLTCLVKGFYPSDIAVEWESNGQPENNYKTTPPVLDSDGSFFLYSRLTVDKSRWQ | |
| | IgG4 CH3 | |
| 241 | EGNVFSCSVMHEALHNHYTQKSLSLSLGK | (SEQ ID NO: 89) |

It is well known that two IgG heavy chains are assembled during cellular processing via disulfide bonds between cysteines located in the hinge region to form a homodimer. It is expected that this will also occur between the modified peptides to form the assembled EMP-hinge core mimetibody construct. In addition, it is expected that the intrachain disulfide bond between the two cysteines in the EMP-1 peptide will also form. The expected structure of EMP-Hinge core mimetibody contains two EMP-1 peptides. The spatial arrangement of the peptides at the N-terminus along with the flexibility of adjoining sequences should allow the peptides to form the bioactive dimer.

The activity of EMP-Hinge core mimetibody was first tested in an in vitro bioactivity assay. For this assay, the EPO dependent UT-7/EPO cell line, derived from a patient with acute megakaryoblastic leukemia, was used (Komatsu et al., 1993, Blood, vol. 82 (2), 456-464). These cells undergo programmed cell death 48 to 72 hours after withdraw from media supplemented with rHuEPO. Cells that have been incubated in the absence of rHuEPO for 24 hours can be saved if treated with rHuEPO or an EPOR agonist. EMP-Hinge core mimetibody was added to cells starved without rHuEPO and cell viability was determined 48 hours after treatment using the tetrazolium compound MTS (CellTiter 96 Aqᵤₑₒᵤₛ One Solution, Promega) that is metabolized by living cells to yield a product with an absorbance that can be measured. Results of a typical assay showed the potency of EMP-Hinge core mimetibody on a molar basis to be 500 fold greater than the EMP-1 peptide and 5 fold less than rHuEPO. In addition, these same cells were stimulated with EMP-Hinge core mimetibody and tyrosine phosphorylation patterns visualized by running cell lysate through a polyacrylamide gel. The pattern exhibited by EMP-Hinge core mimetibody was similar to that of rHuEPO, indicating that the mechanism by which EMP-Hinge core mimetibody acts on these cells is like that of rHuEPO.

In vivo studies were done in normal mice to compare the half-life of EMP-Hinge core mimetibody to that of rHuEPO and to compare their effects on erythropoiesis. When mice were dosed equally, EMP-Hinge core mimetibody gave a higher maximal response and the response was prolonged compared to rHuEPO.

The serum concentrations of both rHuEPO and EMP-Hinge core mimetibody were measured by ELISA. The approximate half-life of EMP hinge core mimetibodies was at least several times that of rHuEPO.

It has been shown that mutation of two lysine (L) residues, L234 & L235, in the IgG1 lower hinge region to alanine (A) will abrogate the ability of the immunoglobulin to mediate complement dependent cytotoxicity (CDC) and antibody dependant cellular cytotoxicity (ADCC) (Hezereh et al., 2001, J. Virol., vol. 75 (24), 12161-68). Preliminary studies have shown that EMP-Hinge core mimetibody does not mediate complement lysis of cells that express the EPO receptor. This may be due to the low number of receptors that are found on erythroid progenitor cells. In addition the in vivo expansion of erythroid progenitors as evidenced by significant increases in hematocrit supports the possible functional irrelevance of immune effector functions. However, while no effector function associated affects have been observed, there remains an interest in introducing these mutations as a precautionary step.

Another modification that would result in a decrease in mediation of immune effector functions is the removal of the glycosylation attachment site. This can be accomplished by mutation of the asparagine at position 297 (N297) to glutamine (Q). Additional changes can optionally include replacing the threonine (T) with an alternative amino acid to reduce or modify O-glycosylation, e.g., T34 or T47 with Aglycosylated versions of the IgG1 subclass are known to be poor mediators of immune effector function (Jefferis et al. 1998, 1 mmol. Rev., vol. 163, 50-76).

Advantages: The novel construct, EMP-Hinge core mimetibody described above offers an alternative way of displaying the bioactive peptide EMP-1. The activity of this construct is in the range of rHuEPO and the in vivo half-life is similar to that of an IgG. In addition, proposed modifications are expected to, in combination and in addition to the novel features of EMP-Hinge core mimetibody, enhance the utility of the EMP-Hinge core mimetibody construct.

### Example 4

### Data Supporting Use of Hinge Deleted EPO Mimetibody in the Treatment of Glucose Intolerance and/or Renal Disease Related Anemia

Advantages: The novel construct, EMP-NfusCG1 described above offers an alternative way of displaying the bioactive peptide EMP-1. The activity of this construct is in the range of rHuEPO and the in vivo half-life is similar to that of an IgG. In addition, proposed modifications are expected to, in combination and in addition to the novel features of EMP-NfusCG1, enhance the utility of the EMP-NfusCG1 construct.
Background: A number of clinical studies indicate that patients with end-stage renal disease often have insulin resistance (Mak., 1996; Spaia et al., 2000; Tuzcu et al., 2004) that could be attributed to uremic toxins, anemia, or secondary hyperparathyroidism (Spaia et al., 2000). Interestingly, treatment with EPO in these patient populations has been shown to improve insulin resistance with a concomitant improvement in blood triglyceride, total cholesterol and LDL levels (Mak., 1996; Spaia et al., 2000). One group suggested that the improvement in insulin sensitivity could be attributed to the EPO itself and not to the correction of the anemia (Spaia et al., 2000).

Recently, Thomas et al., (2005) published a study where 722 patients were screened for diabetic complications and for their EPO levels. Of these patients, approximately 23% had anemia. The authors concluded that the failure to produce EPO in response to declining hemoglobin levels is a common contributor to anemia in patients with diabetes. They also concluded that the lack of EPO production might contribute to diabetic kidney disease.

CNTO 530 is an EPO mimetic that incorporates an EPO-mimetic peptide (EMP-1) to a domain that includes the Fc portion of an antibody. EMP-1 has no sequence homology with EPO but it competes with ¹²⁵I-labeled EPO for binding to the EPO receptor. CNTO 530 also induces proliferation of EPO-responsive cells and stimulates the same intracellular phosphorylation pattern as EPO. CNTO 530 differs from the parent molecule, CNTO 528, in that the EMP-1 peptide is engrafted onto an IgG4 scaffold (rather than IgG1). The scaffold lacks the V-domain and has significantly shorter hinge and linker regions, relative to CNTO 528. The hinge of CNTO 530 incorporates three point mutations (Ala/Ala and S228P) that are lacking in the parent molecule. The respective roles of these changes are to reduce the perceived problem of effector function and to stabilize the molecule. The resulting molecule has significantly improved pharmacokinetic and pharmacodynamic characteristics in rodents and monkeys compared to CNTO 528. Consequently, the sustained in vivo activity of CNTO 530 provides the potential for improved properties compared to EPO and other EPO analogues.

The data from the literature suggest that intervention with EPO could be beneficial to a large number of diabetic patients with declining kidney function. The data presented here show that a long-acting EPO mimetic, such as CNTO 530, could also be beneficial to diabetic patients with declining kidney function. However, CNTO 530 has the additional property of a rather long half-life. Once a month dosing with CNTO 530 would be a novel way to treat diabetic patients suffering from anemia.

### REFERENCES

Mak R H, et al., J. Pediatrics, 129:97-104, 1996.
Spaia S, et al., Nephron, 84:320-325, 2000.
Thomas M C, et al., Arch, Int. Med., 165:466-469, 2005.
Tuzcu A, et al., Horm. Metab. Res., 36:716-720, 2004.

### Example 5

### EPO Mimetibody CNTO 530 Improved Glucose Tolerance Seven Days after Dosing in db/db Mice

Mice (db/db; n=7) were fasted overnight and fasting blood glucose (FBG) was measured. The animals were randomized based upon FBG. The mice were dosed intravenously with CNTO 530 (0.3 mg/kg) and given glucose intraperitonealy (0.5 mg/g) ten minutes later. Blood glucose was measured at various times (10, 20, 30, 60, 90, 120, 150, 180 minutes after glucose). Seven days later, the IPGTT was repeated as described above.

The data shown in FIG. 1 suggest that CNTO 530 had very little effect on glucose tolerance on the first day of the study, but a rather profound effect seven days after a single dose.

### Example 6

### CNTO 530 Improved Glucose Tolerance Seven Days after Dosing in DIO Mice

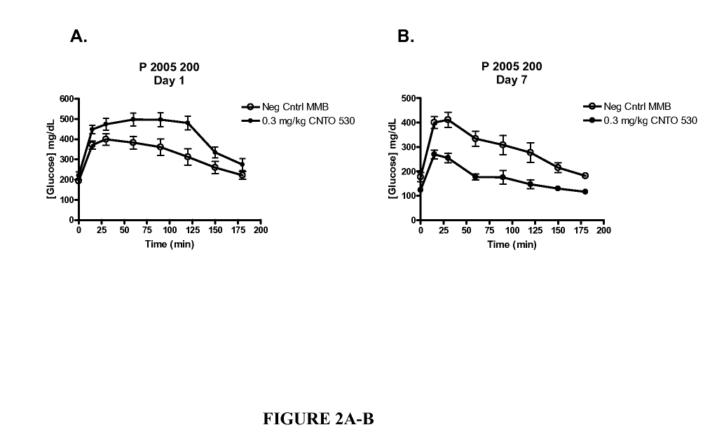

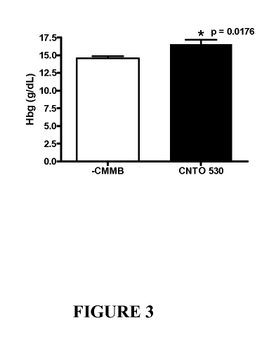

The experiment described in Example 1 was repeated in DIO (diet induced obese) mice following dosing of CNTO 530 (0.3 mg/kg). This murine model is believed to be more representative of the human disease since the mice become diabetic after being fed a high fat diet (Purina TestDiet #58126 consisting of 60.9% kcal fat and 20.8% kcal carbohydrates). As described in Example 1, an IPGTT was done on the day of dosing and seven days after dosing. The time points during the IPGTT were 15, 30, 60, 90, 120, 150, and 180 minutes after glucose challenge. After the IPGTT was completed on day 7, the mice were sacrificed and whole blood (in EDTA) was collected via cardiac puncture for hematology studies. FIG. 2 shows that CNTO 530 improved the glucose tolerance seven days after dosing, but not immediately (10 minutes) after dosing. FIG. 3 indicates that the mice treated with CNTO 530 had elevated hemoglobin relative to the control animals.

### Detailed Description

### Example 7

### A Single Dose of CNTO 530 Improves Glucose Tolerance 7, 14, 21, and 28 Days after Dosing

Mice (DIO; n=7) were fasted overnight and fasting blood glucose (FBG) was measured. The animals were randomized based upon FBG. The mice were dosed intravenously with CNTO 530 (0.3 mg/kg) and given glucose intraperitonealy (0.5 mg/g) ten minutes later. Blood glucose was measured at various times (10, 20, 30, 60, 90, 120, 150, 180 minutes after glucose). The IPGTTs were repeated 7, 14, 21, 28, and 35 days after that initial single dose. Animals in the groups treated for 21, 28, and 35 days were sacrificed after the IPGTT and whole blood was collected for hematology measurements. Following the hematology, the blood was centrifuged and plasma was collected for insulin measurements.

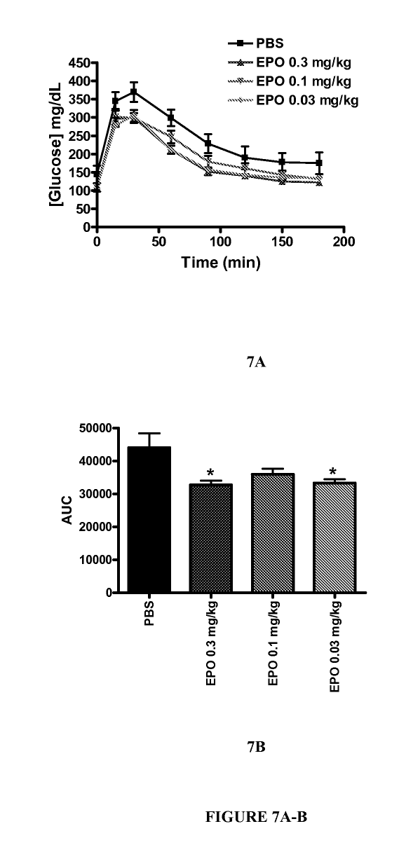

There was a significant reduction in glucose clearance after 14, 21, and 28 days in the animals treated with CNTO 530 relative to the control animals (FIG. 7). Hemoglobin levels were elevated in the treated animals after 21 and 28 days (not tested earlier). Interestingly, insulin levels were significantly decreased in the treated animals after 21 days. Since the glucose levels were so much lower 21 days after dosing, it is not surprising that insulin levels were lower. However, this indicates that the observed phenomenon is real and not an artifact of the glucose measurement.

### Example 8

### A Single Dose of EPO Improves Glucose Tolerance 5 Days after Dosing

Mice (DIO; n=7) were fasted overnight and fasting blood glucose (FBG) was measured. The animals were randomized based upon FBG and dosed intravenously with EPO (0.03, 0.1, 0.3 mg/kg). An IPGTT (as described above) was done 5 days after dosing. The animals were sacrificed after the IPGTT and whole blood was collected for hematology measurements.

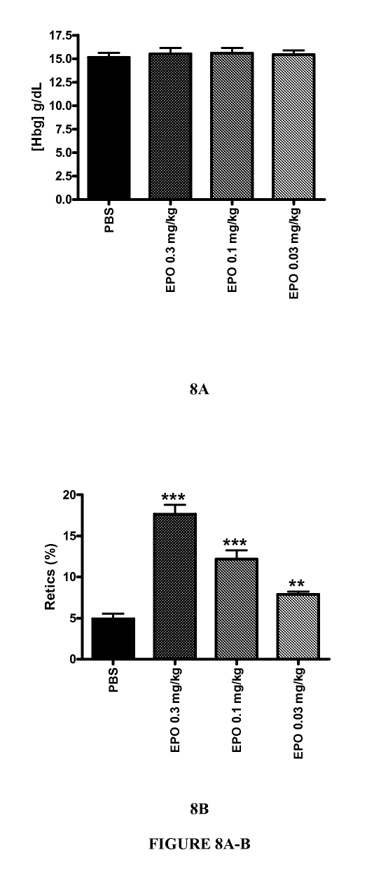

There was a modest improvement in glucose tolerance in the animals dosed with 0.03 and 0.3 mg/kg EPO (FIG. 7) although no statistically significant reduction in fasting blood glucose was observed. FIG. 8 shows that hemoglobin was not significantly elevated but reticulocytes were significantly increased in a dose dependent manner.

### Example 9

### A Single Dose of Darbepoetin Improves Glucose Tolerance 7 Days after Dosing

Mice (DIO; n=7) were fasted overnight and fasting blood glucose (FBG) was measured. The animals were randomized based upon FBG and dosed intravenously with darbepoetin (0.01, 0.03, 0.1 mg/kg). An IPGTT (as described above) was done 7 days after dosing. The animals were sacrificed after the IPGTT and whole blood was collected for hematology measurements.

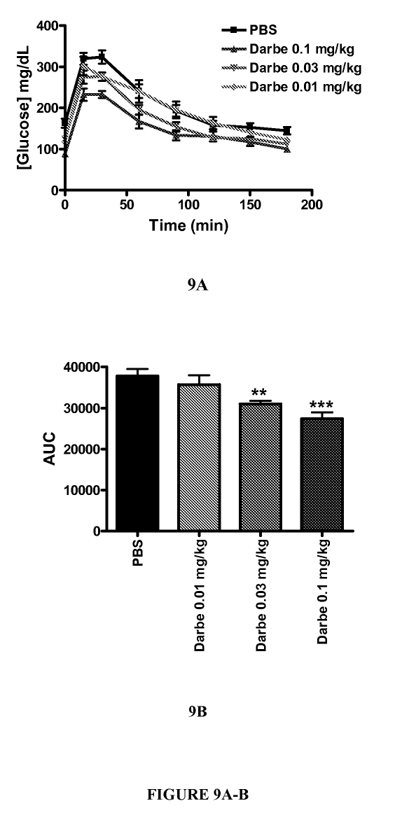

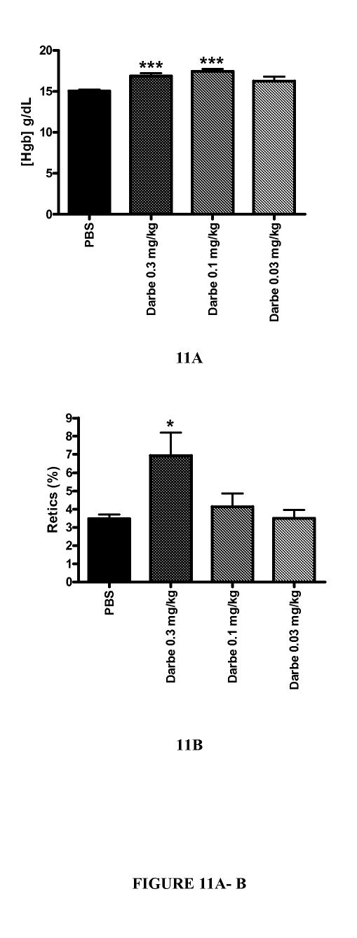

There was a dose dependent improvement in glucose tolerance in the animals dosed with darbe (FIG. 9), and a statistically significant reduction in fasting blood glucose was observed (FIG. 10). FIG. 11 shows that hemoglobin and reticulocytes were significantly elevated.

### Example 10

### IPGTT in EPO Transgenic Mice

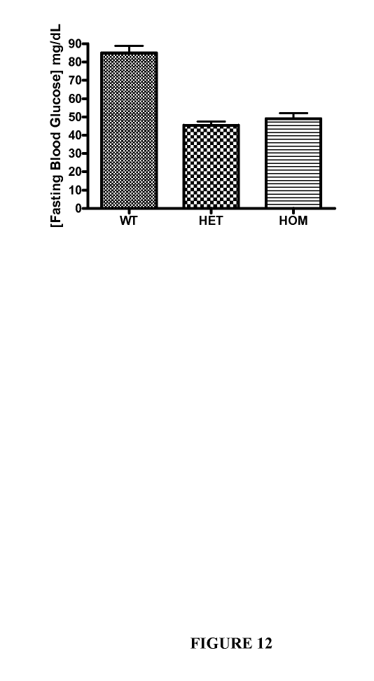

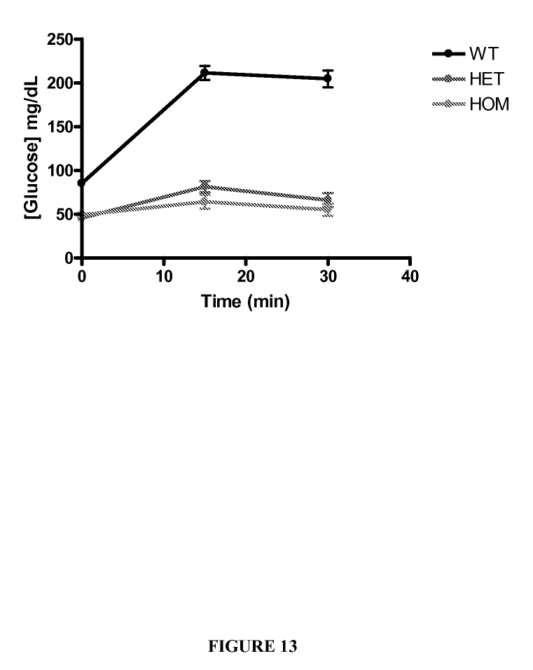

Transgenic mice (heterozygous or homozygous) expressing human EPO and C57B16 mice (age matched to transgenics) were fasted overnight. Fasting blood glucose was measured the next morning and the mice were given glucose intraperitonealy (1.0 mg/g). Blood glucose was measured at various times (10, 20, 30 minutes after glucose). FIG. 12 shows that fasting blood glucose was significantly lower in the transgenic animals relative to the controls. In addition, the area under the curve, during the glucose tolerance test was profoundly improved in the transgenic animals (FIG. 13).

### Example 11

### A Single Dose of CNTO 530 Improves Insulin Sensitivity

Mice (DIO; n=7) were fasted overnight and fasting blood glucose (FBG) was measured. The animals were randomized based upon FBG and dosed intravenously with CNTO 530 (0.3 mg/kg). Fourteen days after dosing, the mice were fasted overnight and blood was collected for glucose and insulin measurements. The animals were dosed intraperitonealy with glucose and blood was collected for glucose and insulin measurements after 20 minutes.

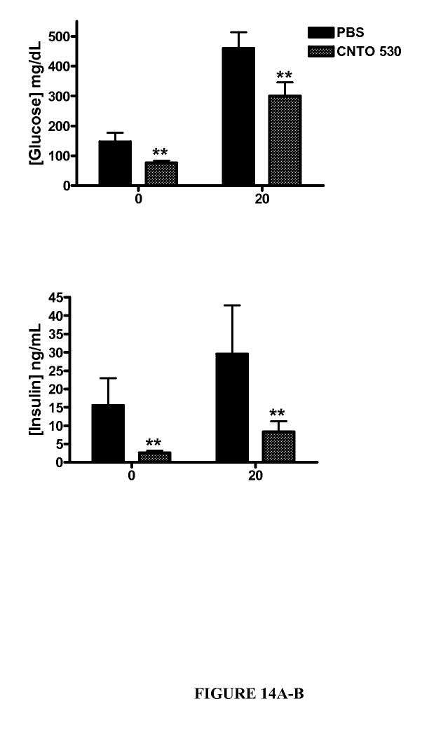

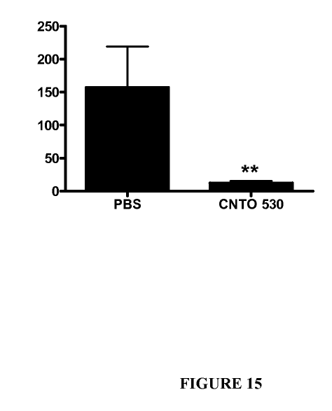

FIG. 14 shows that there was a significant reduction in glucose and insulin levels immediately after the fast and 20 minutes after the glucose challenge. HOMA analysis was used, and the treated animals showed more than a 10-fold reduction in HOMA, suggesting that insulin sensitivity was improved (FIG. 15).

### Example 12

### Datamining Clinical Data

### Summary:

GE Healthcare's Clinical Data Services database contains de-identified, normalized, longitudinal, patient-level Electronic Medical Record data from nearly to 4,000 US based physicians practicing in ambulatory setting who use GE Centricity EMR system. This database contains data on >4.7M patient lives. Average follow up time is approximately 3 years. The database contains information on Patient demographics, medical history, visit type, clinical observations (including Diagnosis codes), laboratory test results and medications prescribed. JNJ has purchased a license to this dataset and mined it to look for outcomes on diabetic parameters in patients who were prescribed EPO for anemia.

Initial mining exercise indicated that CDS database contains a total of 4,700,156 patients of whom 4,331,836 are classified as active patients. Population was further filtered to identify patients with Pre-EPO measurements followed by post-EPO measurements on the following variables of interest
Blood glucose, fasting
Hemoglobin A1c as % of total hemoglobin
Hemoglobin, blood
Hematocrit, blood

Patients with at least 1 pre- (up to 60 days prior) and 1 post- (up to 6 months after) EPO treatment measurements were considered for analysis. Number of patients meeting this criterion varied in numbers for different variables and time zones. Patients who were on EPO medication and also had mean HbA1c value of 6.0 or higher were considered as diabetic.

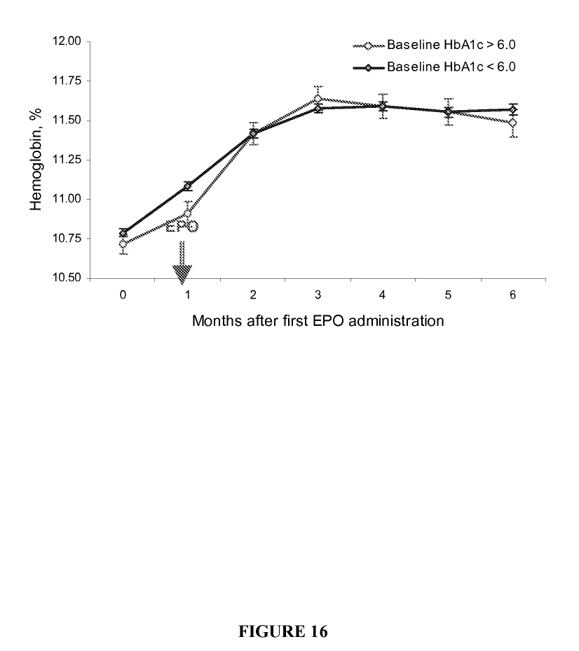

FIG. 16 shows the effect of EPO on the patient's Hemoglobin level. As expected the Hemoglobin level increases steadily over a 3 month period after initial EPO administration before reaching a plateau.

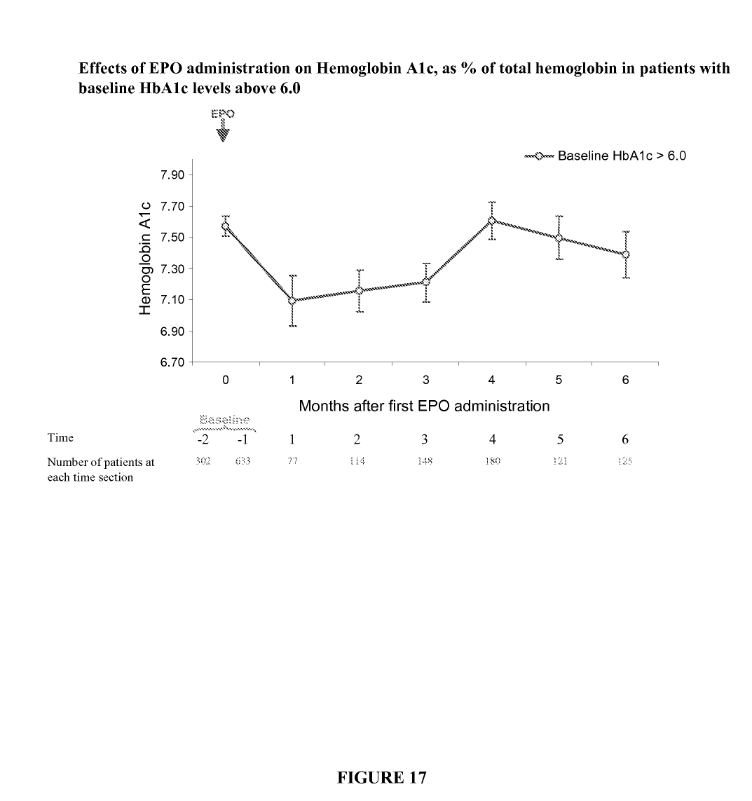

FIG. 17 shows the effect of EPO on patients HbA1C level. HbA1C levels drop by an average value of 0.5% in the first month after EPO administration and remain low in the subsequent two months before bouncing back to the original levels. Comparing FIGS. 16 and 17 it appears that the effect of EPO on HbA1C is independent of its impact on overall Hemoglobin level since FIG. 16 only shows a modest increase in Hemoglobin in the first month after initial EPO administration.

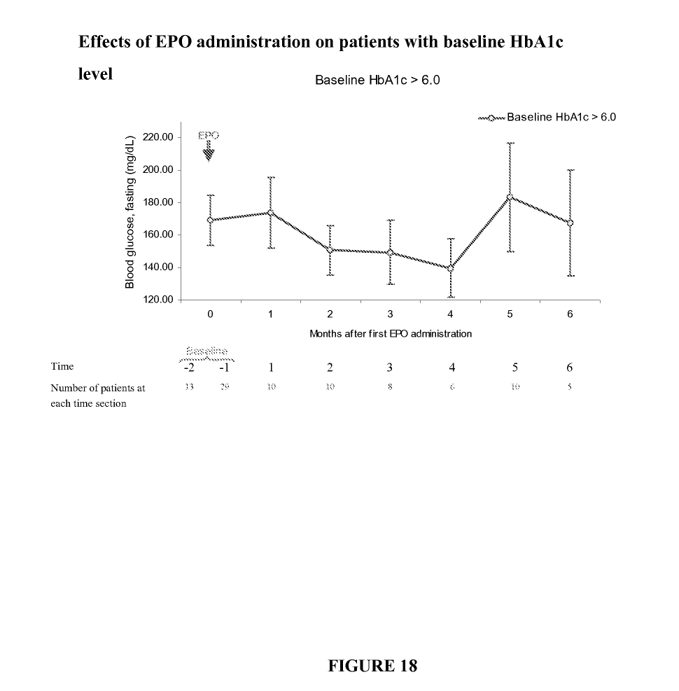

FIG. 18 shows a decrease in fasting blood glucose concentration of approximately 30 mg.dL after initial EPO administration reaching a maximum decline over a 4 month period before reverting back to the original range. The declining in Fasting Blood glucose appears to be more sustained and longer lasting compared to the decline in HbA1C shown in FIG. 17.

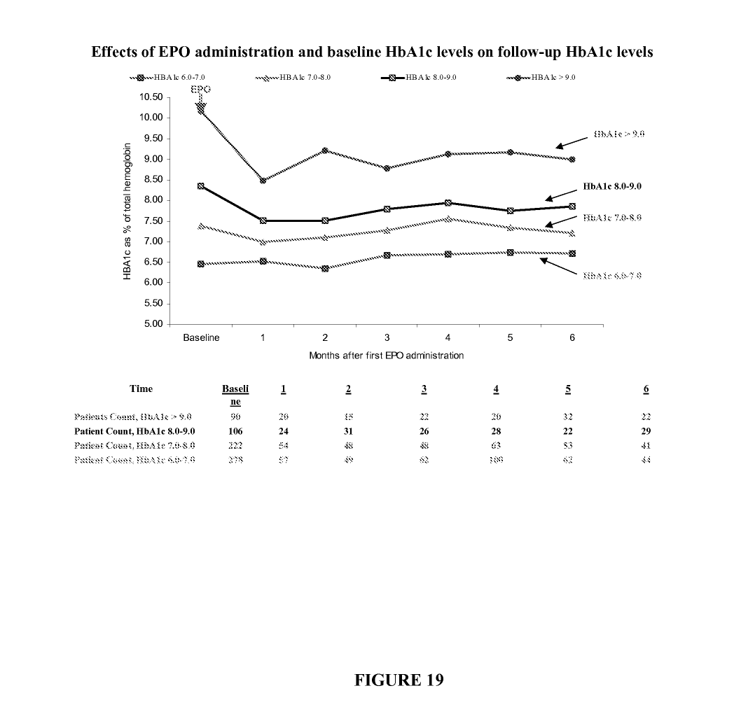

FIG. 19 shows the effect of EPO's effect on HbA1C levels as a function of severity of baseline diabetes. Diabetic population in the database was graded at 4 baseline HbA1C levels: 6-7%, 7-8%, 8-9% and >9%. The figure shows that the ant-diabetic benefit of EPO (defined as the magnitude of decline in HbA1C level) appears to be the greatest in the population that has the highest disease severity (i.e Baseline HbA1C >9%) resulting in approximately 1.69% (10.18 to 8.49%) decline during the first month. In contrast starting with a more moderate level of disease (i.e. Baseline HbA1C=8%-9% and 7%-8%) the anti-diabetic effect was also lower, 0.84% (8.35% to 7.51%) and 0.39% (7.38% to 6.99%), respectively. There was no decline observed in the population at the lowest baseline level (6.0%-7.0%).

One of the disadvantages of EMR data tends to be missing/incomplete observations along a time course. In order to assure ourselves that we are indeed observing longer term therapeutic effect in diabetes we further mined the database to isolate only those patients treated with EPO for whom we have baseline HbA1C values present in the 2 month period prior to initial EPO administration and who also have HbA1C values in the two subsequent time sections (1-3 months and 4-6 months). The results obtained with this longitudinal dataset confirm the observations reported in FIG. 19. For baseline HBA1C values >8 the magnitude of HbA1C decline was 0.95% (9.41% to 8.46%) within the first 3 months after EPO administration. The effect plateaued with no further decline observed in the next 3 month period. At a lower baseline HbA1C value (7-8%) the amount of decline in the first 3 months was 0.46% (7.47% to 7.01%) followed by no significant further change in the next 3 months.

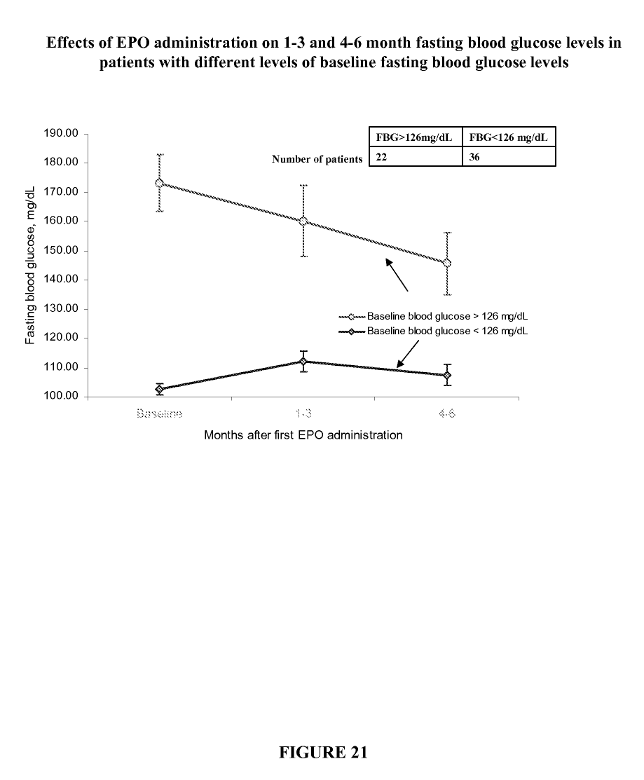

Analysis using longitudinal patients was repeated for fasting blood glucose data after initial EPO administration. FIG. 21 shows the effect on fasting blood glucose on patients with baseline value either greater than or below 126 mg/dL (Baseline fasting blood glucose level of 126 mg/dL was used as a criteria to diagnose diabetes patients as suggested by American Diabetes Association (ADA). Results show a steady decline on Fasting blood glucose values over the entire time course for diabetic patients (FBG>126 mg/dL) averaging 28 mg/dL (173 mg/dL to 145 mg/dL). Patients with baseline value under 126 mg/dL do not show any decline in FBG level. Instead they show a slight increase at the end of the time course equivalent to 5 mg/dL (102 mg/dL to 107 mg/dL).

The data presented suggest that diabetic patients could be treated with an EPO receptor agonist to improve their fasting blood glucose and insulin sensitivity. The data shown in FIGS. 7 and 8 and FIGS. 16-18 suggest that an increase in hemoglobin may not be required in order to improve glucose tolerance, suggesting that it may be possible to use a low dose of an ERA to treat the diabetes without increasing hemoglobin levels. This is significant since ERAs are known to increase the risk of thrombosis at levels that significantly raise hemoglobin. The clinical data also suggests that this is possible. The clinical data showed that a dose of EPO that allowed a modest improvement in hemoglobin (<1%) had significant improvements in fasting blood glucose and HbA1c.

Advantages: The use of an ERA as a therapeutic to treat glucose intolerance in diabetic patients could provide several advantages.
This therapy could provide a novel mechanism of action for treatment of diabetes.
This therapy could lead to dual treatment of anemia and diabetes.
Due to the improvements in insulin sensitivity, an improvement in lipids (HDL, LDL, TG) is also possible with an ERA.
The sustained effect on hyperglycemia could result in a significant improvement in HbA1c and delay of islet b cell loss in diabetic patients.

Current therapy for Type 2 diabetes requires at least daily dosing. Treatment of this patient population with an ERA may allow for less frequent dosing and therefore improved compliance.

The use of EPO receptor agonists with extended half lifes, such as CNTO 530, as a therapeutic to treat anemia and glucose intolerance in renal disease patients could provide several advantages over other EPO receptor agonists (ERAs). This therapy is expected to be useful for dual treatment of anemia and diabetes. The extended half-life of EPO receptor agonists with extended half lifes, such as CNTO 530, compared to other ERAs is expected to be useful for treating hyperglycemia in diabetic renal failure patients. The sustained effect on hyperglycemia is expected to be useful for treating islet β cell loss by delaying or reducing this process. The extended half-life of CNTO 530 results in less frequent dosing compared to other ERAs. The lack of homology between CNTO 530 and EPO reduces the possibility of PRCA in this patient population. The glucose tolerizing effect could minimize the requirement of this patient group for additional diabetes drugs.

It will be clear that the invention can be practiced otherwise than as particularly described in the foregoing description and examples.

Numerous modifications and variations of the present invention are possible in light of the above teachings and, therefore, are within the scope of the present invention

1. A method for treating glucose intolerance in a cell, tissue, organ or animal, comprising contacting or administering a composition comprising an effective amount, to said cell, tissue, organ or animal, of at least one erythropoietin (EPO) receptor agonist.

2. A method according to claim 1, wherein said EPO receptor agonist is selected from an EPO receptor agonist biologic and a EPO receptor agonist small molecule compound.

3. A method according to claim 2, wherein said EPO receptor agonist biologic is selected from a polypeptide, an antibody and an antibody fusion polypeptide.

4. A method according to claim 3, wherein said polypeptide is EPO or a naturally occurring variant.

5. A method according to claim 3, wherein said polypeptide further comprises at least one polyethylene glycol molecule.

6. A method according to claim 3, wherein said polypeptide is a non-naturally occurring variant of EPO.

7. A method according to claim 5, wherein said non-naturally occurring variation of EPO is darbepoetin-alfa.

8. A method according to claim 3, wherein said antibody is an agonist antibody to the EPO receptor or to EPO.

9. A method according to claim 3, wherein said polypeptide is a functional mimetic of EPO.

10. A method according to claim 9, wherein said functional mimetic of EPO is at least one selected from SEQ ID NOS:1-30.

11. A method according to claim 3, wherein said polypeptide is hematide.

12. A method according to claim 3, wherein said antibody fusion polypeptide comprises at least a portion of a heavy chain antibody sequence and at least one EPO receptor agonist polypeptide.

13. A method according to claim 12, wherein said polypeptide is an EPO functional mimetic.

14. A method according to claim 2, wherein said small molecule is a chemical compound that is an EPO receptor agonist.

15. A method according to claim 14, wherein said small molecule is selected from FG-2216 and FG-4592.

16. A method according to claim 1, wherein said effective amount is 0.001-50 mg of said EPO receptor agonist is 0.000001-500 mg.

17. A method according to claim 15, wherein said contacting or said administrating is by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

18. A method according to claim 1, further comprising administering, prior, concurrently or after said (a) contacting or administering, at least one composition comprising an effective amount of at least one compound or polypeptide selected from at least one of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an ophthalmic, otic or nasal drug, a topical drug, a nutritional drug, a cytokine, or a cytokine antagonist.


SEQUENCE LISTING (89 sequences)

SEQ ID NO: 1 - protein, 14 aa, Artificial Sequence
  Synthetic peptide
YXCXXGPXTWXCXP

SEQ ID NO: 2 - protein, 20 aa, Artificial Sequence
  Synthetic peptide
GGTYSCHFGPLTWVCKPQGG

SEQ ID NO: 3 - protein, 20 aa, Artificial Sequence
  Synthetic peptide
GGDYHCRMGPLTWVCKPLGG

SEQ ID NO: 4 - protein, 20 aa, Artificial Sequence
  Synthetic peptide
GGVYACRMGPITWVCSPLGG

SEQ ID NO: 5 - protein, 20 aa, Artificial Sequence
  Synthetic peptide
VGNYMCHFGPITWVCRPGGG

SEQ ID NO: 6 - protein, 20 aa, Artificial Sequence
  Synthetic peptide
GGLYLCRFGPVTWDCGYKGG

SEQ ID NO: 7 - protein, 20 aa, Artificial Sequence
  Synthetic peptide
GGTYSCHFGPLTWVCKPQGG

SEQ ID NO: 8 - protein, 23 aa, Artificial Sequence
  Synthetic peptide
GGTYSCHFGPLTWVCKPQGGSSK

SEQ ID NO: 9 - protein, 10 aa, Artificial Sequence
  Synthetic peptide
CXXGPXTWXC

SEQ ID NO: 10 - protein, 19 aa, Artificial Sequence
  Synthetic peptide
GGTYSCHGPLTWVCKPQGG

SEQ ID NO: 11 - protein, 19 aa, Artificial Sequence
  Synthetic peptide
VGNYMAHMGPITWVCRPGG

SEQ ID NO: 12 - protein, 18 aa, Artificial Sequence
  Synthetic peptide
GGPHHVYACRMGPLTWIC

SEQ ID NO: 13 - protein, 18 aa, Artificial Sequence
  Synthetic peptide
GGTYSCHFGPLTWVCKPQ

SEQ ID NO: 14 - protein, 20 aa, Artificial Sequence
  Synthetic peptide
GGLYACHMGPMTWVCQPLRG

SEQ ID NO: 15 - protein, 22 aa, Artificial Sequence
  Synthetic peptide
TIAQYICYMGPETWECRPSPKA

SEQ ID NO: 16 - protein, 13 aa, Artificial Sequence
  Synthetic peptide
YSCHFGPLTWVCK

SEQ ID NO: 17 - protein, 11 aa, Artificial Sequence
  Synthetic peptide
YCHFGPLTWVC

SEQ ID NO: 18 - protein, 10 aa, Artificial Sequence
  Synthetic peptide
XXXGPXTWXX

SEQ ID NO: 19 - protein, 12 aa, Artificial Sequence
  Synthetic peptide
YXXXXGPXTWXX

SEQ ID NO: 20 - protein, 14 aa, Artificial Sequence
  Synthetic peptide
XYXXXXGPXXXXXX

SEQ ID NO: 21 - protein, 18 aa, Artificial Sequence
  Synthetic peptide
XYXCXXGPXTWXCXXXLL

SEQ ID NO: 22 - protein, 20 aa, Artificial Sequence
  Synthetic peptide
GGLYLCRFGPVTWDCGYKGG

SEQ ID NO: 23 - protein, 20 aa, Artificial Sequence
  Synthetic peptide
GGTYSCHFGPLTWVCKPQGG

SEQ ID NO: 24 - protein, 20 aa, Artificial Sequence
  Synthetic peptide
VGNYMCHFGPITWVCRPGGG

SEQ ID NO: 25 - protein, 20 aa, Artificial Sequence
  Synthetic peptide
GGVYACRMGPITWVCSPLGG

SEQ ID NO: 26 - protein, 22 aa, Artificial Sequence
  Synthetic peptide
TIAQYICYMGPETWECRPSPKA

SEQ ID NO: 27 - protein, 13 aa, Artificial Sequence
  Synthetic peptide
YSCHFGPLTWVCK

SEQ ID NO: 28 - protein, 11 aa, Artificial Sequence
  Synthetic peptide
YCHFGPLTWVC

SEQ ID NO: 29 - protein, 12 aa, Artificial Sequence
  Synthetic peptide
SCHFGPLTWVCK

SEQ ID NO: 30 - protein, 12 aa, Artificial Sequence
  Synthetic peptide
AXXXXGPXTWXX

SEQ ID NO: 31 - protein, 125 aa, Homo Sapiens
  VARIANT 32, 47, 80: Xaa = Any Amino Acid
QVQLLVQSGAEVKKPGASVKVSCKASGYTFTXWVRQAPGQGLEWMGXRVTMTRDTSTSTAYMELSSLRSEDTAVYYCARXWGQGTLVTVSSGSTKGPSVFPLAPSSKSTSGGTAALGCLVKDYFP

SEQ ID NO: 32 - protein, 124 aa, Homo Sapiens
  VARIANT 31, 46, 79: Xaa = Any Amino Acid
QITLKESGPALVKPTQTLTLTCTFSGFSLSXWIRQPPGKALEWLAXRLTITKDTSKNQVVLTMTNMDPVDTATYYCARXWGQGTLVTVSSASPTSPKVFPLSLSSKSTSGGTAALGCLVKDYFP

SEQ ID NO: 33 - protein, 100 aa, Homo Sapiens
  VARIANT 32, 47, 80: Xaa = Any Amino Acid
EVVQLVESGGGLVQPGGSLRLSCAASGFTFSXWVRQAPGKGLEWVSXRFTISRDNSKNTLYLQMNSLRAEDTAVYYCARXWGQGTLVTVSSGSTKAPSVF

SEQ ID NO: 34 - protein, 102 aa, Homo Sapiens
  VARIANT 31, 46, 79: Xaa = Any Amino Acid
EVQLVESGGGLVKPGGSLRLSCAASGFTFSXWVRQAPGKGLEWVGXRFTISRDDSKNTLYLQMNSLKTEDTAVYYCTTXWGQGTLVTVSSASTKGPSVFPLA

SEQ ID NO: 35 - protein, 101 aa, Homo Sapiens
  VARIANT 31, 46, 80: Xaa = Any Amino Acid
EVQLVESGGGLVQPGRSLRLSCTASGFTFGXWVRQAPGKGLEWVGXRFTISRDDSKSIAYLQMNSLKTEDTAVYYCTRNXWGQGTLVTVSSGSTKGPSVLP

SEQ ID NO: 36 - protein, 108 aa, Homo Sapiens
  VARIANT 34, 49, 82: Xaa = Any Amino Acid
QVQLQESGPGLVKPSETLSLTCTVSGGSSISSSXWIRQPPGKGLEWIGXRVTISVDTSKNQFSLKLSSVTAADTAVYYCARXWGQGTLVTVSSAPTKAPDVFPIISGC

SEQ ID NO: 37 - protein, 132 aa, Homo Sapiens
  VARIANT 32, 47, 80: Xaa = Any Amino Acid
EEVQLVQSGAEVKKPGESLKISCKGSGYSFTXWVRQMPGKGLEWMGXQVTISADKSISTAYLQWSSLKASDTAMYYCARXWGQGTLVTVSSASTKAPSVFPLVSCENSPSDTSSVAVGCLAQDFLPDSITFS

SEQ ID NO: 38 - protein, 125 aa, Homo Sapiens
  VARIANT 31, 46, 79: Xaa = Any Amino Acid
QVQLQQSGPGLVKPSQTLSLTCAISGDSVSXWIRQSPSRGLEWLGXRITINPDTSKNQFSLQLNSVTPEDTAVYYCARXWGQGTLVTVSSGSASAPTLFPLVSCENSPSDTSSVAVGCLAQDFLP

SEQ ID NO: 39 - protein, 91 aa, Homo Sapiens
  VARIANT 31, 46, 79: Xaa = Any Amino Acid
QVQLVQSGSELKKPGASVKVSCKASGYTFTXWVRQAPGQGLEWMGXRFVFSLDTSVSTAYLQISSLKAEDTAVYYCARXWGQGTLVTVSSS

SEQ ID NO: 40 - protein, 93 aa, Homo Sapiens
  VARIANT 25, 41, 74: Xaa = Any Amino Acid
DIQMTQSPSSLSASVGDRRVTITCXWYQQKPGKAPKLLIYXGVPSRFSGSGSGTDFTLTISSLQPEDFATYYCXFGQGTKVEIKRTVAAPSVF

SEQ ID NO: 41 - protein, 92 aa, Homo Sapiens
  VARIANT 24, 40, 73: Xaa = Any Amino Acid
DIVMTQSPLSLPVTPGQPASISCXWYLQKPGQSPQLLIYXGVPDRFSGSGSGTDFTLKISRVEAEDVGVYYCXFGQGTKVEIKRTVAAPSVF

SEQ ID NO: 42 - protein, 91 aa, Homo Sapiens
  VARIANT 24, 40, 73: Xaa = Any Amino Acid
EIVLTQSPGTLSLSPGERATLSCXWYQQKPGQAPRLLIYXGIPDRFSGSGSGTDFTLTISRLEPEDFAVYYCXFGQGTKVEIKRTVAAPSV

SEQ ID NO: 43 - protein, 85 aa, Homo Sapiens
  VARIANT 24, 40, 73: Xaa = Any Amino Acid
ETTLTQSPAFMSATPGDKVNISCXWYQQKPGEAAIFIIQXGIPPRFSGSGYGTDFTLTINNIESEDAAYYFCXLRHFWPGDQAAG

SEQ ID NO: 44 - protein, 79 aa, Homo Sapiens
  VARIANT 18, 34, 67: Xaa = Any Amino Acid
EIVMTQSPVNLSMSAGEXWYQQKPGQAPRLFIYXGISARFSGSGSGTDFTLTITSLQSEDFAVYYCXFGQGTKLDIKRT

SEQ ID NO: 45 - protein, 77 aa, Homo Sapiens
  VARIANT 16, 32, 65: Xaa = Any Amino Acid
ELTQSPGTLSLSPGEXWYQHKPGQAPRLVIHXGISDRFSGSGSGTDFTLTITRLEPEDFALYYCXFGQGTKLDFKRT

SEQ ID NO: 46 - protein, 95 aa, Homo Sapiens
  VARIANT 25, 41, 74: Xaa = Any Amino Acid
DIVMTQSPDSLAVSLGGRRATINCXWYQQKPGQPPKLLIYXGVPDRFSGSGSGTDFTLTISSLQAEDVAVYYCXFGGGTKVEIKRTVAAPSVFKF

SEQ ID NO: 47 - protein, 98 aa, Homo Sapiens
  VARIANT 23, 39, 72: Xaa = Any Amino Acid
QSVLTQPPSVSGAPGQRVTISCXWYQQLPGTAPKLLIYXGVPDRFSGSKSGTSASLAISGLQSEDEADYYCXFGGGTKLTVLGQPKAAPSVTLFPPSS

SEQ ID NO: 48 - protein, 99 aa, Homo Sapiens
  VARIANT 24, 40, 73: Xaa = Any Amino Acid
AQSVLTQPPSVSAAPGQKVTISCXWYQQLPGTAPKLLIYXGIPDRFSGSKSGTSATLGITGLQTGDEADYYCXFGGGTKLTVLGQPKAAPSVTLFPPSS

SEQ ID NO: 49 - protein, 99 aa, Homo Sapiens
  VARIANT 23, 39, 72: Xaa = Any Amino Acid
QSALTQPASVSGSPGQSITISCXWYQQHPGKAPKLMIYXGVSNRFSGSKSGNTASLTISGLQAEDEADYYCXFGGGTTKLTVLGQPKAAPSVTLFPPSS

SEQ ID NO: 50 - protein, 107 aa, Homo Sapiens
  VARIANT 23, 39, 72: Xaa = Any Amino Acid
SYELTQPPSVSVSPGQTARITCXWYQQKPGQAPVLVIYXGIPERFSGSSSGTTATLTISGVQAEDEADYYCXFGGGTKLTVLGQPKAAPSVTLFPPSSEELQANKAT

SEQ ID NO: 51 - protein, 93 aa, Homo Sapiens
  VARIANT 23, 40, 73: Xaa = Any Amino Acid
SYVLTQPPSVSVAPGQTARITCXWYQQKPGQAPVLVVYDXGIPERFSGSNSGNTATLTISRVEAGDEADYYCXFGGGTKLTVLGQPKAAPTVT

SEQ ID NO: 52 - protein, 98 aa, Homo Sapiens
  VARIANT 23, 39, 72: Xaa = Any Amino Acid
SYELTQPPSVSVSPGQTASITCXWYQQKPGQSPVLVIYXGIPERFSGSNSGNTATLTISGTQAMDEADYYCXFGGGTKLTVLGQPKAAPSRSLCPPPP

SEQ ID NO: 53 - protein, 98 aa, Homo Sapiens
  VARIANT 23, 39, 72: Xaa = Any Amino Acid
SSELTQDPAVSVALGQTVRITCXWYQQKPGQAPVLVIYXGIPDRFSGSSSGNTASLTITGAQAEDEADYYCXFGGGTKLTVLGQPKAAPSVTLFPPSS

SEQ ID NO: 54 - protein, 94 aa, Homo Sapiens
  VARIANT 23, 39, 72: Xaa = Any Amino Acid
QPVLTQSSSASASLGSSVKLTCXWHQQQPGKAPRYLMKXGVPDRFSGSSSGADRYLTISNLQSEDEADYYCXFGGGTKLTVLGQPKAAPSVTLF

SEQ ID NO: 55 - protein, 95 aa, Homo Sapiens
  VARIANT 23, 39, 72: Xaa = Any Amino Acid
QLVLTQSPSASASLGASVKLTCXWHQQQPEKGPRYLMKXGIPDRFSGSSSGAERYLTISSLQSEDEADYYCXFGGIGGGTKLTVLGQPKAAPSVS

SEQ ID NO: 56 - protein, 88 aa, Homo Sapiens
  VARIANT 23, 40, 75: Xaa = Any Amino Acid
QAVLTQPSSLSASPGASASLTCXWYQQKPGSPPQYLLRYXGVPSRFSGSKDASANAGILLISGLQSEDEADYYCXFGGGTKLTVLSQP

SEQ ID NO: 57 - protein, 101 aa, Homo Sapiens
  VARIANT 23, 39, 74: Xaa = Any Amino Acid
NFMLTQPHSVSESPGKTVTISCXWYQQRPGSAPTTVIYXGVPDRFSGSIDSSSNSASLTISGLKTEDEADYYCXFGGGTKLTVLGQPKAAPSVTLFPPSSS

SEQ ID NO: 58 - protein, 89 aa, Homo Sapiens
  VARIANT 23, 39, 72: Xaa = Any Amino Acid
QAVVTQEPSLTVSPGGTVTLTCXWFQQKPGQAPRALIYXWTPARFSGSLLGGKAALTLSGVQPEDEAEYYCXFGGGTKLTVLGQPKAAP

SEQ ID NO: 59 - protein, 89 aa, Homo Sapiens
  VARIANT 23, 39, 72: Xaa = Any Amino Acid
QTVVTQEPSFSVSPGGTVTLTCXWYQQTPGQAPRTLIYXGVPDRFSGSILGNKAALTITGAQADDESDYYCXFGGGTKLTVLGQPKAAP

SEQ ID NO: 60 - protein, 91 aa, Homo Sapiens
  VARIANT 23, 39, 72: Xaa = Any Amino Acid
QPVLTQPPSASASLGASVTLTCXWYQQRPGKGPRFVMRXGIPDRFSVLGSGLNRYLTIKNIQEEDESDYHCXFGGGTKLTVLGQPKAAPSV

SEQ ID NO: 61 - protein, 87 aa, Homo Sapiens
  VARIANT 23, 39, 72: Xaa = Any Amino Acid
QAGLTQPPSVSKGLRQTATLTCXWLQQHQGHPPKLLSYXGISERFSASRSGNTASLTITGLQPEDEADYYCXFGGGTKLTVLGQPKA

SEQ ID NO: 62 - protein, 354 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSZEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 63 - protein, 340 aa, Homo Sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 64 - protein, 384 aa, Homo Sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 65 - protein, 497 aa, Homo Sapiens
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPVGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKDVCVEEAEGEAPWTWTGLCIFAALFLLSVSYSAALTLLMVQRFLSATRQGRPQTSLDYTNVLQPHA

SEQ ID NO: 66 - protein, 339 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESBNGQPENNYKTTPPVLDSDGSFFLYSKLTVDKSRWQQGNVFSCSVMHEALHNHYTQKSLSLSPGKTHTCPPCP

SEQ ID NO: 67 - protein, 326 aa, Homo Sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 68 - protein, 377 aa, Homo Sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 69 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 70 - protein, 476 aa, Homo Sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRSKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWQMQRGQPLSPEKYVTSAPMPEPQAPGRYFAHSILTVSEEEWNTGETYTCVVAHEALPNRVTERTVDKSTGKPTSADEEGFENLWATASTFIVLYNVSLVMSDTAGTCYVK

SEQ ID NO: 71 - protein, 107 aa, Homo Sapiens [sequence omitted]

SEQ ID NO: 72 - protein, 107 aa, Homo Sapiens
GQPKAAPSVTLFPPSSEELQANKATLVCLISDFYPGAVTVAWKADSSPVKAGVETTTPSKQSNNKYAASSYLSLTPEQWKSHRKSYSCQVTHEGSTVEKTVAPTECS

SEQ ID NO: 73 - protein, 4 aa, Artificial Sequence
  Synthetic peptide
GGGS

SEQ ID NO: 74 - protein, 6 aa, Artificial Sequence
  Synthetic peptide
GSGGGS

SEQ ID NO: 75 - protein, 5 aa, Artificial Sequence
  Synthetic peptide
CPPCP

SEQ ID NO: 76 - protein, 110 aa, Artificial Sequence
  Synthetic peptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 77 - protein, 110 aa, Artificial Sequence
  Synthetic peptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 78 - protein, 107 aa, Artificial Sequence
  Synthetic peptide
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 110 aa, Artificial Sequence
  Synthetic peptide
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 80 - protein, 110 aa, Artificial Sequence
  Synthetic peptide
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 81 - protein, 107 aa, Artificial Sequence
  Synthetic peptide
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 247 aa, Artificial Sequence
  Synthetic peptide
QIQGGTYSCHFGPLTWVCKPQGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 249 aa, Artificial Sequence
  Synthetic peptide
QIQGGTYSCHFGPLTWVCKPQGGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 251 aa, Artificial Sequence
  Synthetic peptide
QIQGGTYSCHFGPLTWVCKPQGGGSGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 247 aa, Artificial Sequence
  Synthetic peptide
QIQGGTYSCHFGPLTWVCKPQGGGSCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 249 aa, Artificial Sequence
  Synthetic peptide
QIQGGTYSCHFGPLTWVCKPQGGGGGSCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 247 aa, Artificial Sequence
  Synthetic peptide
QIQGGTYSCHFGPLTWVCKPQGGGSCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - protein, 247 aa, Artificial Sequence
  Synthetic peptide
QIQGGTYSCHFGPLTWVCKPQGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 249 aa, Artificial Sequence
  Synthetic peptide
QIQGGTYSCHFGPLTWVCKPQGGGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK